(19) United States
(12) Patent Application Publication
(10) Pub. No.: US 2026/0008772 A1
(43) Pub. Date: Jan. 8, 2026

(54) 2-Indolone Derivatives and Use Thereof

(71) Applicant: TIANJIN ZHENGCHENG MEDICAL TECHNOLOGY CO., LTD., Tianjin (CN)

(72) Inventor: Yuna Lu, Tianjin (CN)

(21) Appl. No.: 19/329,636

(22) Filed: Sep. 16, 2025

Related U.S. Application Data
(63) Continuation-in-part of application No. PCT/CN2024/070212, filed on Jan. 2, 2024.

(30) Foreign Application Priority Data
Jan. 3, 2023 (CN) 202310000618.7
Dec. 31, 2023 (CN) 202311860729.1

(51) Int. Cl. C07D 403/12; A61K 31/496; A61P 11/00; C07B 59/00

(52) U.S. Cl. CPC C07D 403/12; A61K 31/496; A61P 11/00; C07B 59/002

(57) ABSTRACT

A 2-indolone derivative and a pharmaceutically acceptable salt thereof is provided. Also provided are a pharmaceutical composition containing a pharmaceutically acceptable carrier, and the 2-indolone derivative and the pharmaceutically acceptable salt thereof. The 2-indolone derivative and the pharmaceutically acceptable salt thereof can be used for preparing a drug for treating and preventing related diseases such as fibrosis and the like, and can also be used for preparing a drug for treating and preventing related diseases such as excessive or abnormal cell proliferation and the like.

### CROSS-REFERENCE TO RELATED APPLICATIONS

The present application is a continuation-in-part application of PCT application No. PCT/CN2024/070212 filed on Jan. 2, 2024, which claims the benefit of Chinese Patent Application Nos. 202310000618.7 filed on Jan. 3, 2023 and 202311860729.1 filed on Dec. 31, 2023. The contents of all of the aforementioned applications are incorporated by reference herein in their entirety.

### TECHNICAL FIELD

The present invention belongs to the field of biomedicine, and specifically relates to a 2-indolone derivative, a pharmaceutical composition containing the compound and application thereof.

### BACKGROUND ART

Nintedanib is an oral small molecule tyrosine kinase inhibitor developed by Boehringer-Ingelheim, which can competitively inhibit receptor tyrosine kinases such as fibroblast growth factor receptor (FGFR 1-3), vascular endothelial growth factor receptor (VEGFR 1-3), platelet-derived growth factor receptor (PDGFRα and β). Nintedanib is used to treat idiopathic pulmonary fibrosis (IPF); for the treatment of systemic sclerosis-related interstitial lung disease (SSc-ILD); Nintedanib is used to treat chronic fibrotic interstitial lung disease (ILD) with a progressive phenotype, and can also be used to treat cancer.

Currently, nintedanib still has poor absorption, distribution, metabolism and/or excretion (ADME) performance, which hinders its wider use or limits its application in specific indications. For example, due to the low oral bioavailability of the drug and/or the short elimination half-life in the body, the exposure of the drug in the body is relatively small. The commonly used solution is to administer the drug frequently or at high doses to obtain a sufficiently high level of drug exposure. However, this introduces a large number of potential treatment problems, such as patient compliance with medication intervals, and higher doses, more severe side effects, and increased treatment costs.

In order to obtain better ADME and/or pharmacological properties and achieve better therapeutic benefits, drugs or drug candidates can be structurally modified. Due to the complexity of biological systems, the changes in the ADME and/or pharmacological properties of drugs or drug candidates caused by structural modifications are usually unpredictable and require empirical research.

In addition, deuterated modification is also an option. Compared with hydrogen, deuterium forms stronger chemical bond with carbon. In selected cases, the bond strength of the increase given by deuterium can positively affect the ADME performance of medicine, has the potentiality of improving drug effect, safety and/or tolerability. Simultaneously, because the size and shape of deuterium are similar to hydrogen, compared with the original chemical entity that only comprises hydrogen, it is expected that replacing hydrogen with deuterium will not affect the biochemical efficacy and selectivity of medicine.

However, due to the complex metabolic process of biological systems, the pharmacokinetic properties of drugs in vivo are affected by many factors and also show corresponding complexity. Compared with the corresponding non-deuterated drugs, the changes in the pharmacokinetic properties of deuterated drugs show great randomness and unpredictability. For some compounds, deuterium substitution slows down their metabolic clearance in the body and increases their half-life; for other compounds, deuterium substitution does not cause metabolic changes; for still other compounds, deuterium substitution accelerates metabolic clearance and shortens their half-life (Blake, M I et al, J Pharm Sci, 1975, 64: 367-91; Foster, AB, Adv Drug Res 1985, 14: 1-40; Kushner, D J et al, Can J Physiol Pharmacol 1999, 79-88; Fisher, M B et al, Curr Opin Drug Discov Dev, 2006, 9: 101-09; Scott L. Harbeson, Roger D. Tung. Deuterium in Drug Discovery and Development, P405-406).

In order to overcome the problems of nintedanib, the present invention obtains a class of drugs with good ADME performance, good pharmacological activity, reduced dosage or frequency of use, and/or reduced toxic side effects by structural modification and/or deuteration modification of nintedanib.

### SUMMARY OF THE INVENTION

The object of the present invention is to provide 2-indolone derivatives and uses thereof.

The present invention first provides a compound shown in formula I, or a pharmaceutically acceptable salt thereof:

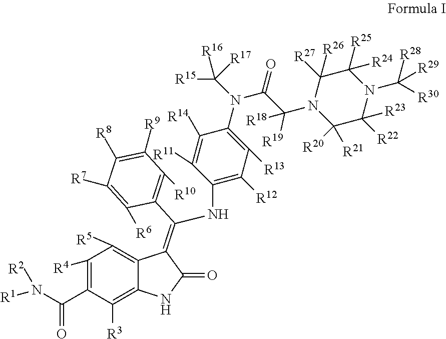

wherein:
R¹ and R² are each independently selected from H, deuterium (D), C₁₋₄ alkyl, C₂₋₄ alkenyl, C₂₋₄ alkynyl, 3-6-membered saturated or unsaturated carbocyclic ring, 3-6-membered saturated or unsaturated heterocyclic ring containing nitrogen (N) and/or oxygen (O) and/or sulphur (S), wherein the C₁₋₄ alkyl, C₂₋₄ alkenyl, C₂₋₄ alkynyl, 3-6-membered saturated or unsaturated carbocyclic ring, 3-6-membered saturated or unsaturated heterocyclic ring containing nitrogen (N) and/or oxygen (O) and/or sulphur (S) are optionally substituted by zero or more substituents selected from deuterium, halogen, cyano, hydroxyl, sulfhydryl; or R¹ and R², together with the atoms to which they are attached, form a ring;
R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are each independently selected from hydrogen (H) or deuterium (D);
The conditions are R³, R⁴, R¹, R⁶, R⁷, R¹, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are all hydrogen, the R¹ or R² group contains at least one deuterium atom.

Further, the present invention provides the following fourteen compounds (Compound ND801, Compound ND802, Compound ND803, Compound ND804, Compound ND805, Compound ND806, Compound ND807, Compound ND808, Compound ND809, Compound ND810, Compound ND811, Compound ND812, Compound ND813 or Compound ND814) or any one of their optical isomers, pharmaceutically acceptable salts, hydrates, solvates or prodrugs.

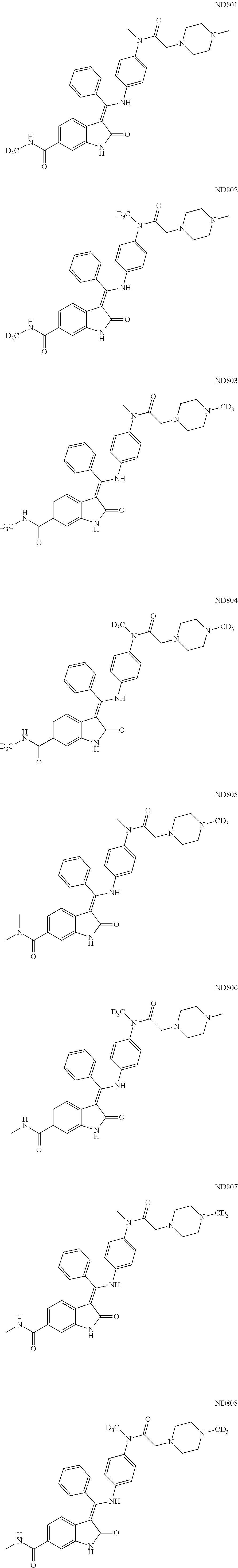

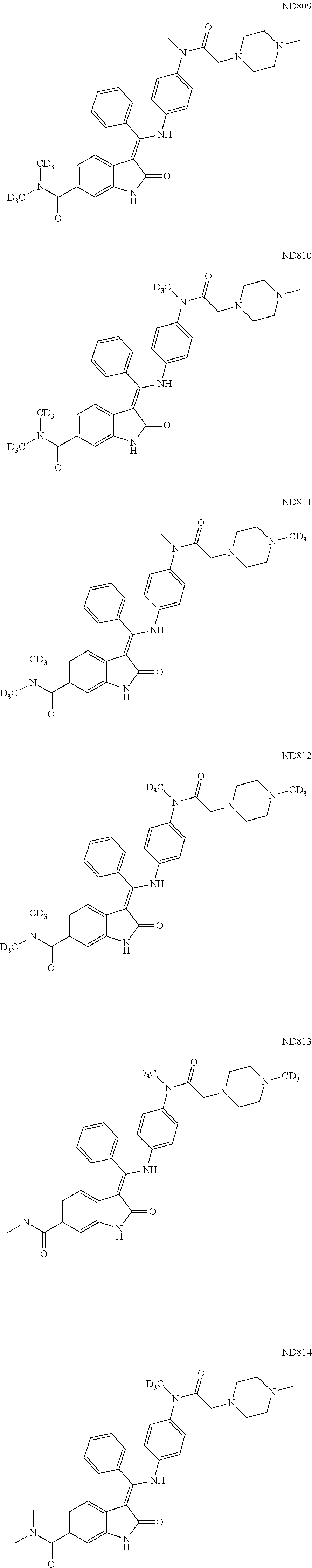

Furthermore, the pharmaceutically acceptable salt is a methanesulfonate, ethanesulfonate, toluenesulfonate, benzenesulfonate, phosphate, dextrorotatory camphorsulfonate, hydrochloride, hydrobromide, hydrofluoride, sulfate, nitrate, formate, acetate, propionate, oxalate, malonate, succinate, fumarate, maleate, lactate, malate, tartrate, citrate, picrate, aspartate or glutamate of the compound, preferably an ethanesulfonate.

The present invention also provides the use of the aforementioned compound or its optical isomers, pharmaceutically acceptable salts, hydrates, solvates or prodrugs in the preparation of drugs for treating or preventing fibrosis-related diseases.

Furthermore, the fibrosis-related diseases are idiopathic pulmonary fibrosis, giant cell interstitial pneumonia, sarcoidosis, cystic fibrosis, respiratory distress syndrome, drug-induced pulmonary fibrosis, granulomatosis, scleroderma, interstitial lung disease, pneumoconiosis, silicosis, asbestosis, acute lung injury, cardiac fibrosis, cirrhosis, chronic kidney disease, myocardial infarction, heart failure, non-alcoholic fatty liver disease (NASH), COVID-19, and lupus erythematosus.

The present invention also provides the use of the aforementioned compound or its optical isomers, pharmaceutically acceptable salts, hydrates, solvates or prodrugs in the preparation of drugs for treating and preventing diseases related to excessive or abnormal cell proliferation.

Furthermore, the diseases associated with excessive or abnormal cell proliferation include cancer, preferably the following diseases: acute myeloid leukemia, gastric tumors, neuroendocrine tumors, thyroid tumors, melanoma, squamous cell carcinoma, metastatic non-small cell lung cancer, soft tissue sarcoma, pterygium; neovascular eye disease.

The present invention also provides the use of the aforementioned compound or its optical isomers, pharmaceutically acceptable salts, hydrates, solvates or prodrugs in the preparation of VEGFR inhibitors or FGFR inhibitors or PDGFR inhibitors. VEGFR refers to vascular endothelial growth factor receptor. FGFR refers to fibroblast growth factor receptor. PDGFR refers to platelet-derived growth factor receptor.

The present invention also provides a therapeutic drug, which is a preparation prepared by using the aforementioned compound or its optical isomer, pharmaceutically acceptable salt, hydrate, solvate or prodrug as an active ingredient and adding pharmaceutically acceptable excipients. The present invention also provides a combined drug, which is composed of the aforementioned compound or its optical isomer, pharmaceutically acceptable salt, hydrate, solvate or prodrug and other therapeutic drugs in any proportion.

As used herein, “deuterated” refers to one or more hydrogens (H) in a compound being replaced by deuterium (D). In a preferred embodiment, the deuterium isotope content of deuterium at the deuterium substituted position is greater than the natural deuterium isotope content (0.015%), more preferably greater than 40%, more preferably greater than 70%, more preferably greater than 90%, more preferably greater than 95%, more preferably greater than 99%, and more preferably greater than 99.5%.

As used herein, the term “pharmaceutically acceptable salt” refers to a salt suitable for use as a drug formed by a compound of the present invention and an acid or base. Pharmaceutically acceptable salts include inorganic salts and organic salts. A preferred class of salts is a salt formed by a compound of the present invention and an acid, including but not limited to: methanesulfonate, ethanesulfonate, benzenesulfonate, benzenesulfonate, phosphate, right-rotating camphorsulfonate, hydrochloride, hydrobromide, hydrofluoride, sulfate, nitrate, formate, acetate, propionate, oxalate, malonate, succinate, fumarate, maleate, lactate, malate, tartrate, citrate, picrate, aspartate or glutamate. Furthermore, the pharmaceutically acceptable salt is ethanesulfonate.

The pharmaceutically acceptable excipient has certain physiological activity, but the addition of the component will not change the dominant position of the above-mentioned drug in the treatment process of the disease, but only play an auxiliary effect, which is only the utilization of the known activity of the component and is a common auxiliary treatment method in the medical field. If the above-mentioned auxiliary components are used in combination with the drugs of the present invention, they should still fall within the scope of protection of the present invention.

### The Positive Improvement Effect of the Present Invention is

(1) The compounds of the present invention have good pharmacokinetic properties, can reduce dosage and/or reduce toxic metabolites, and have better druggability.
(2) The compounds of the present invention have good effects in preventing and treating organ fibrosis, and can be effectively used for the treatment of pulmonary fibrosis, liver fibrosis, cardiac fibrosis, kidney fibrosis, and/or other organ fibrosis-related diseases.
(3) The compounds of the present invention have good therapeutic and preventive effects on diseases related to excessive or abnormal cell proliferation, and can be effectively used for the treatment of acute myeloid leukemia, gastric tumors, neuroendocrine tumors, thyroid tumors, melanoma, squamous cell carcinoma, metastatic non-small cell lung cancer, soft tissue sarcoma, pterygium and/or neovascular eye disease.
(4) The compounds of the present invention have good VEGFR, FGFR, and/or PDGFR inhibitory effects and can be effectively used for diseases associated with VEGFR, FGFR, and/or PDGFR.

### BRIEF DESCRIPTION OF THE DRAWINGS

FIG. 1 is a schematic diagram of the comparison of hydroxyproline (HYP) content in the right lung of mice, wherein a: control group, b: model group, c: nintedanib group, d: example compound group, and the ordinate represents the HYP content.

FIG. 2 is a schematic diagram of the results of FVC measurement in mice, wherein a: control group, b: model group, c: nintedanib group, d: example compound group, and the ordinate represents the FVC value.

FIG. 3 is a schematic diagram of the results of mouse Cdyn determination, wherein a: control group, b: model group, c: nintedanib group, d: example compound group, and the ordinate represents the Cdyn value.

FIG. 4 is a schematic diagram of the results of mouse Re measurement, wherein a: control group, b: model group, c: nintedanib group, d: example compound group, and the ordinate represents the Re measurement value.

FIG. 5 is a schematic diagram of the Ri measurement results of mice, wherein a: control group, b: model group, c: nintedanib group, d: example compound group, and the vertical ordinate represents the Ri measurement value.

FIG. 6 is a HE staining image of mouse pulmonary fibrosis, wherein a: control group, b: model group, c: nintedanib group, d: example compound group.

FIG. 7 is a schematic diagram of the measurement of pulmonary fibrosis area in mice, wherein b: model group, c: nintedanib group, d: example compound group, and the ordinate represents the percentage of fibrosis area.

### DETAILED DESCRIPTION OF THE EMBODIMENTS

The following examples further illustrate the invention. It should be understood that these examples are only for illustrating the invention and do not limit the scope of the invention.

Experimental methods without specific conditions in the following examples are generally carried out under conventional conditions or according to the conditions recommended by the manufacturer.

The raw materials and instruments used in the present invention can be purchased from the market.

Taking compound ND803 as an example, a preferred preparation process is as follows:

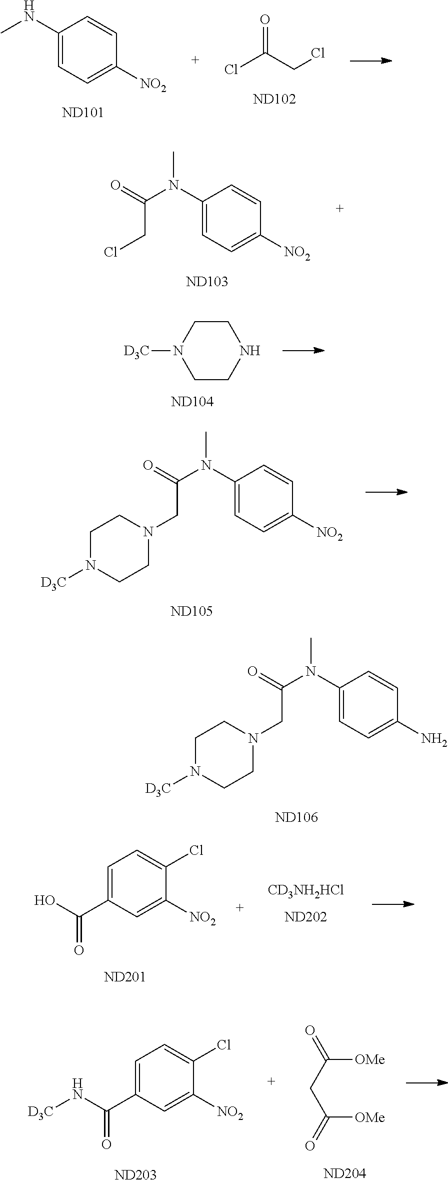

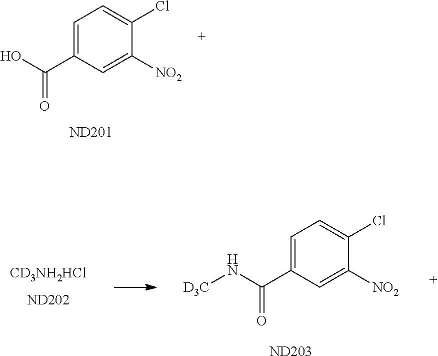

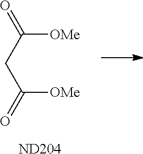

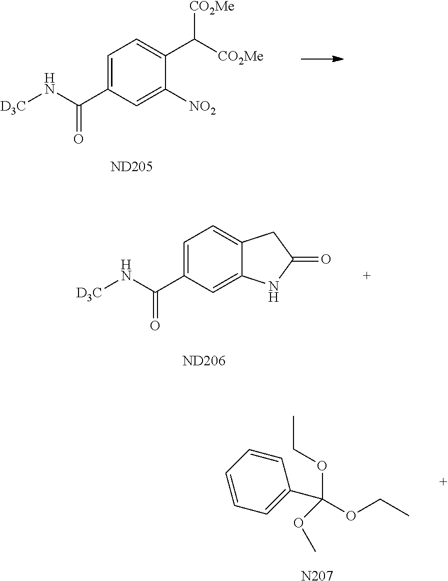

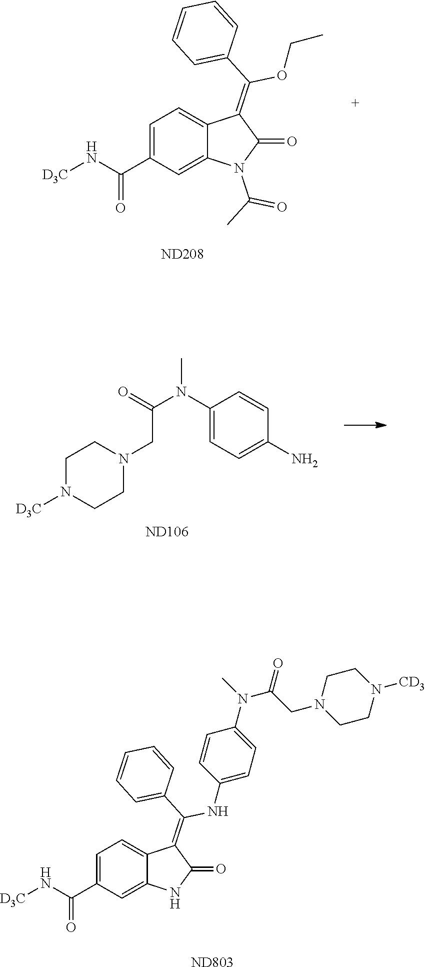

The specific synthesis method is described in Example 1.

Taking compound ND801 as an example, another preferred preparation process is as follows:

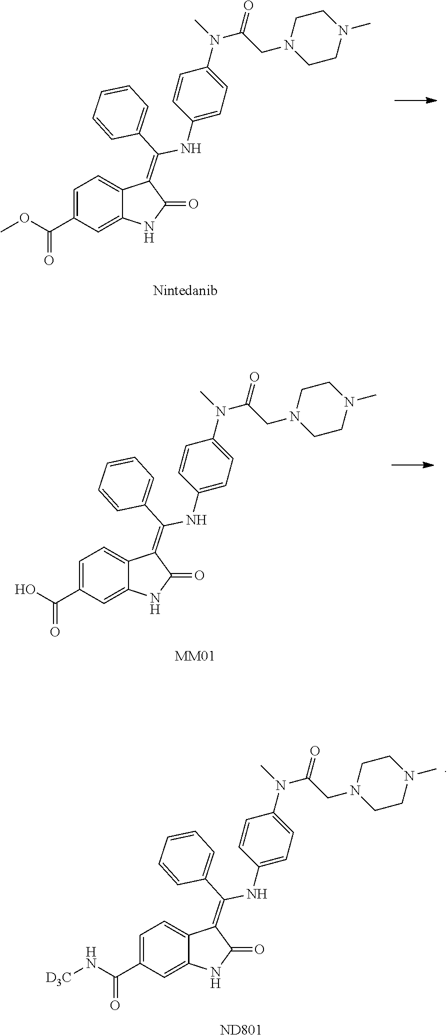

The specific synthesis method is described in Examples 2 and 3.

### Example 1: Synthesis of Compound ND803

### Step 1: Synthesis of Compound ND203

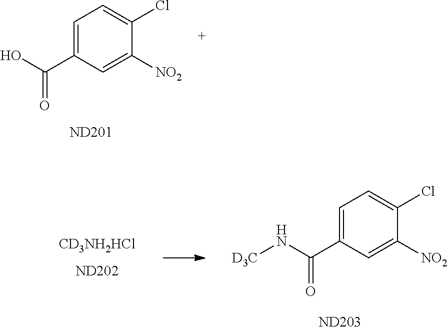

Add 3 mL of ultra-dry N,N-dimethylformamide (DMF) to 2-(7-azabenzotriazole)-N,N,N′,N′-tetramethyluronium hexafluorophosphate (HATU) (380 mg, 1 mmol), stir, and add 2 mL of ultra-dry DMF solution of compound ND201 (202 mg, 1 mmol) and DIEA (661 uL, 4 mmol) at 0° C. After stirring the mixture for 10 min, add trideuterated methylamine hydrochloride (compound ND202) (2 mmol), remove the ice bath, and stir the reaction at room temperature for 24 h. After the reaction is completed, transfer the product to a separatory funnel, add 30 mL of water, and extract with ethyl acetate (3×10 mL). Combine the organic layers, wash with water (3×20 mL) and saturated brine (20 mL) in turn. Add anhydrous sodium sulfate to dry, filter, and concentrate the filtrate under reduced pressure. The crude product was purified by flash column chromatography, eluted with a mixed solvent of dichloromethane and methanol, and concentrated under reduced pressure to obtain compound ND203.

### Step 2: Synthesis of Compound ND205

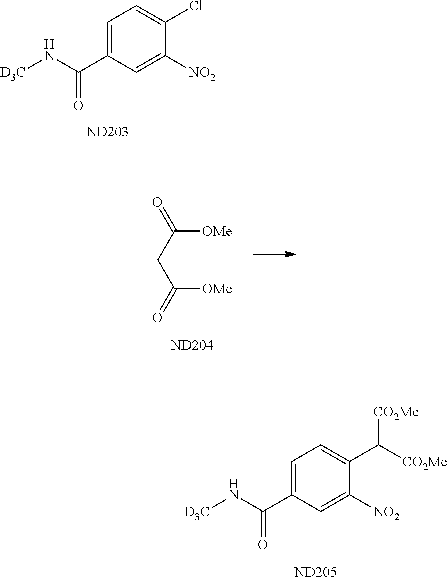

Potassium carbonate (40 mmol) and dimethyl malonate (compound ND204) (22 mmol) were added to anhydrous N,N-dimethyl sulfoxide (DMSO) (30 mL) in sequence, and the mixture was stirred at room temperature for 2 min. Compound ND203 (20 mmol) was added in batches and the mixture was reacted at 40° C. for 3 h. Ethyl acetate (60 mL) was added, and the mixture was washed with 1 mol·L−1 hydrochloric acid (60 mL) and saturated brine (2×60 mL) in sequence, dried over anhydrous magnesium sulfate, concentrated, and the residue was recrystallized with ethyl acetate and dried to obtain compound ND205.

### Step 3: Synthesis of Compound ND206

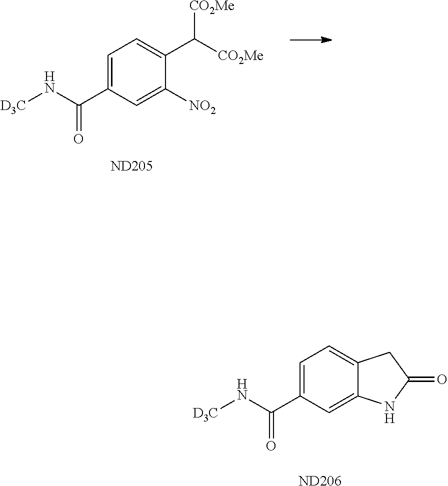

Compound ND205 (7.5 mmol), 5% Pd/C (0.23 g), ammonium formate (75 mmol) and glacial acetic acid (15 mL) were added to the reaction flask in sequence, and the mixture was reacted at 100° C. for 3 h under nitrogen protection. Pd/C was removed by hot filtration, and the filtrate was distilled under reduced pressure. Saturated sodium bicarbonate solution was slowly added to the residual liquid until no bubbles overflowed, and the mixture was stirred for 1 h. The mixture was filtered, and the solid was rinsed with water (15 mL) and dried to obtain compound ND206.

### Step 4: Synthesis of Compound ND208

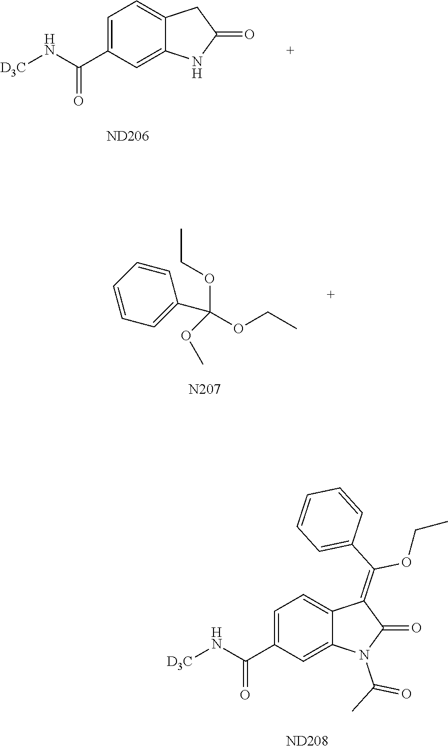

Compound ND206 (6 mmol), toluene (6 mL) and acetic anhydride (3 mL) were added to the reaction flask in sequence, and triethyl orthobenzoate (compound ND207) (18 mmol) was added dropwise under stirring. After the addition was completed, the mixture was refluxed at 110° C. for 2 h. The mixture was cooled to room temperature, concentrated, saturated sodium bicarbonate solution 23 mL was added, stirred for 30 min, extracted with ethyl acetate (2×30 mL), the extracts were combined, washed with saturated sodium bicarbonate solution (2×45 mL) and saturated brine (45 mL) in sequence, dried over anhydrous magnesium sulfate, concentrated, and the residue was recrystallized with petroleum ether and dried to obtain compound ND208.

### Step 5: Synthesis of Compound ND103

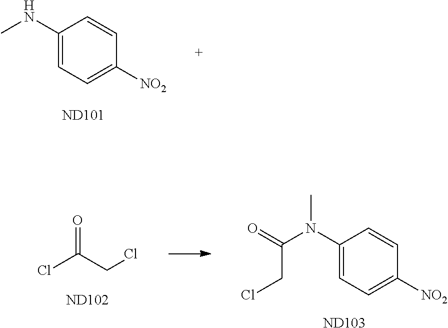

Compound N101 (50 mmol) and potassium carbonate (40 mmol) were added to ethyl acetate (60 mL) in sequence, stirred at room temperature, and compound ND102 (chloroacetyl chloride) (5.62 mL, 75 mmol) dissolved in ethyl acetate (20 mL) was added dropwise, and reacted at room temperature for 1 h. Water (3×60 mL) was added for washing, and the liquid was separated. The organic layer was dried over anhydrous magnesium sulfate, filtered, and the filtrate was concentrated. The obtained solid was recrystallized with ice ethanol and dried to obtain compound ND103.

### Step 6: Synthesis of Compound ND105

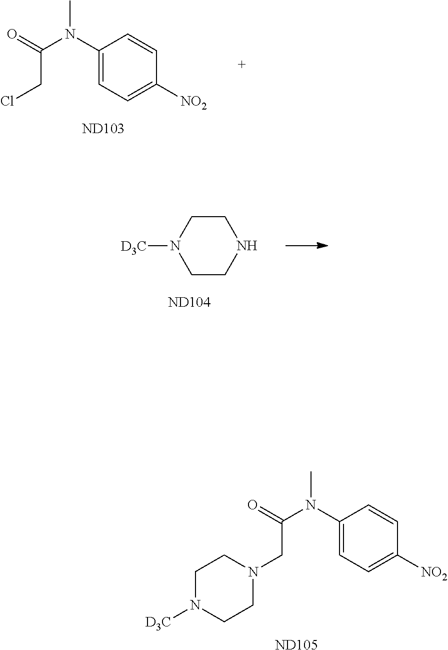

Compound ND103 (30 mmol) and trideuterated methylpiperazine (compound ND104) (33 mmol) were added to acetonitrile (30 mL) in sequence, and the mixture was stirred at 35° C. for 3 h. The mixture was filtered and the filtrate was concentrated. Ethyl acetate (45 mL) was added to the residue, which was washed with saturated brine (3×45 mL) in sequence, dried over anhydrous magnesium sulfate, and concentrated to obtain compound ND105.

### Step 7: Synthesis of Compound ND106

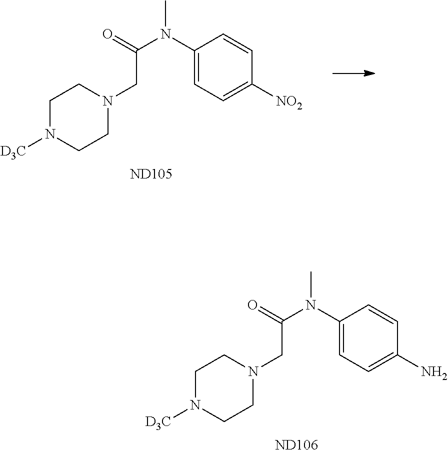

Compound ND105 (25 mmol) and 10% Pd/C (0.75 g) were added to methanol (90 mL) in sequence, and hydrogen was passed through. The mixture was stirred at room temperature for 24 h, Pd/C was filtered out, and an oil was obtained by distillation under reduced pressure. Ether was added to precipitate a solid, which was filtered and the filter cake was dried to obtain compound ND106.

### Step 8: Synthesis of Compound ND803

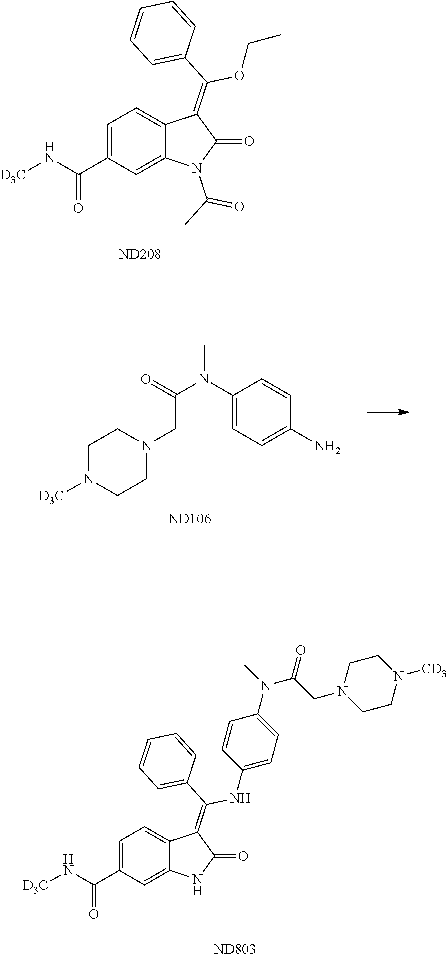

Compound ND208 (3 mmol) and sodium methoxide (6 mmol) were added to methanol (12 mL) in sequence, and the mixture was stirred at room temperature for 2 h. Compound ND106 (3.6 mmol) and dimethyl sulfoxide (6 mL) were added, the temperature was raised to 80° C., and the mixture was reacted for 1 h. Methanol was removed by distillation under reduced pressure, ethyl acetate (30 mL) was added, and the mixture was washed with water (3×30 mL) in sequence, dried over anhydrous magnesium sulfate, concentrated, recrystallized with anhydrous methanol, and dried to obtain compound ND803. The hydrogen nuclear magnetic resonance spectrum of compound ND803 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (8H).

### Example 2: Synthesis of Compound MM01

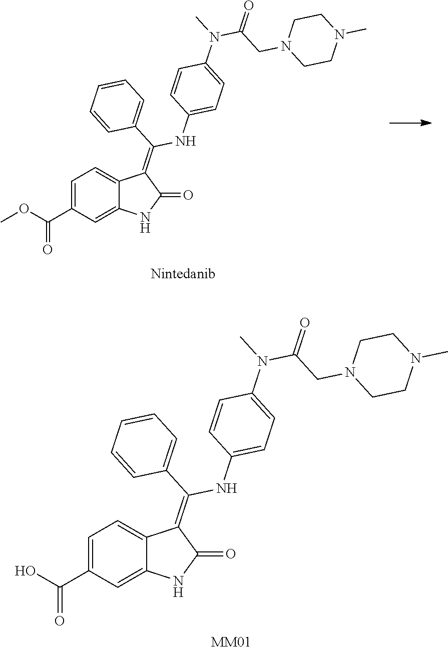

### Step 9: Synthesis of Compound MM01

Nintedanib (1.62 g, 3 mmol) was added to 15 mL of tetrahydrofuran, and then sodium hydroxide solution (1 mol/L, 6 mL) was slowly added, and the mixture was heated to 50° C. and stirred for reaction. The reaction process was detected by TLC. After 8 hours of reaction, the mixture was concentrated under reduced pressure, 10 mL of water was added, and hydrochloric acid solution (1 mol/L) was added dropwise to adjust the pH value to 2-3. A large amount of yellow solid precipitated, and the filter cake was washed with 20 mL of ethanol. The crude product was purified by Flash column chromatography (dichloromethane:methanol=10:1) and concentrated under reduced pressure to obtain compound MM01.

### Example 3: Synthesis Method 2 of Compound ND801

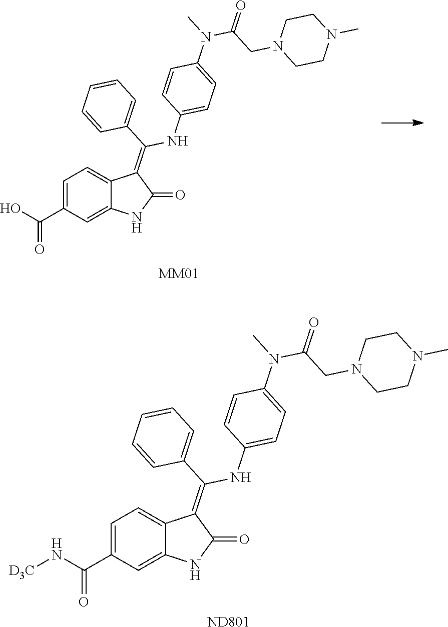

### Step 10: Synthesis of Compound ND801

Add 3 mL of ultra-dry N,N-dimethylformamide (DMF) to 2-(7-azabenzotriazole)-N,N,N′,N′-tetramethyluronium hexafluorophosphate (HATU) (380 mg, 1 mmol), stir, and add 2 mL of ultra-dry DMF solution of MM01 (526 mg, 1 mmol) and DIEA (661 uL, 4 mmol) to the suspension at 0° C. After stirring the mixture for 10 min, add trideuterated methylamine hydrochloride (compound ND202) (2 mmol), remove the ice bath, and stir the reaction at room temperature for 24 h. After the reaction is completed, transfer the product to a separatory funnel, add 30 mL of water, and extract with ethyl acetate (3×10 mL). Combine the organic layers and wash with water (3×20 mL) and saturated brine (20 mL) in turn. Add anhydrous sodium sulfate to dry, filter, and concentrate the filtrate under reduced pressure. The crude product was purified by flash column chromatography (dichloromethane:methanol=7:1) and concentrated under reduced pressure to obtain compound ND801. The hydrogen nuclear magnetic resonance spectrum of compound ND801 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (11H).

### Example 4: Synthesis of Compound ND302

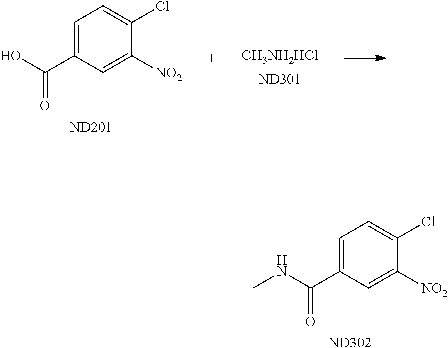

### Step 11: Synthesis of Compound ND302

Add 3 mL of ultra-dry N,N-dimethylformamide (DMF) to 2-(7-azabenzotriazole)-N,N,N′,N′-tetramethyluronium hexafluorophosphate (HATU) (380 mg, 1 mmol), stir, and add 2 mL of ultra-dry DMF solution of compound ND201 (202 mg, 1 mmol) and DIEA (661 uL, 4 mmol) to the suspension at 0° C. After stirring the mixture for 10 min, add methylamine hydrochloride (compound ND301) (2 mmol), remove the ice bath, and stir the reaction at room temperature for 24 h. After the reaction is completed, transfer the product to a separatory funnel, add 30 mL of water, and extract with ethyl acetate (3×10 mL). Combine the organic layers, wash with water (3×20 mL) and saturated brine (20 mL) in turn. Add anhydrous sodium sulfate to dry, filter, and concentrate the filtrate under reduced pressure. The crude product was purified by flash column chromatography, eluted with a mixed solvent of dichloromethane and methanol, and concentrated under reduced pressure to obtain compound ND302.

### Example 5: Synthesis of Compound ND304

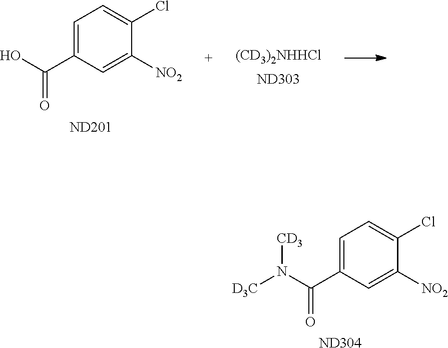

Compound ND301 was replaced by compound ND303, and the reaction was carried out according to the method of “Example 4: Synthesis of compound ND302” to obtain compound ND304.

### Example 6: Synthesis of Compound ND306

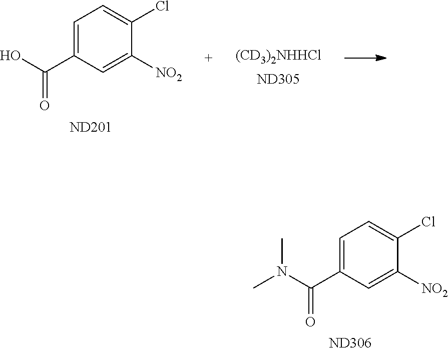

Compound ND301 was replaced by compound ND305, and the reaction was carried out according to the method of “Example 4: Synthesis of compound ND302” to obtain compound ND306.

### Example 7: Synthesis of Compound ND203

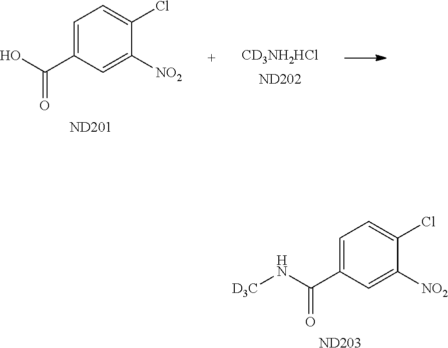

Compound ND202 was used to replace compound ND301, and the reaction was carried out according to the method of “Example 4: Synthesis of compound ND302” to obtain compound ND203.

### Example 8: Synthesis of Compound ND401

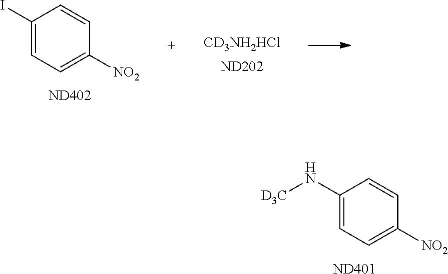

Step 12: Synthesis of compound ND401 Add 4-nitroiodobenzene (compound ND402) (10 mmol), trideuterated methylamine hydrochloride (compound ND202) (50 mmol), 5.3 mL of 9.5 mol/L sodium hydroxide (50 mmol) aqueous solution and copper powder (5 mol %) in a 30 mL sealed tube, stir with magnetic force, react in an oil bath at 100° C. for 12 hours, then cool to room temperature and extract with ethyl acetate three times (3×20 mL). Combine the ethyl acetate extracts, add anhydrous sodium sulfate to dry, filter, and concentrate the filtrate under reduced pressure. The crude product is purified by silica gel column and eluted with a mixed solvent of petroleum ether and ethyl acetate to obtain compound ND401. The hydrogen nuclear magnetic resonance spectrum of compound ND401 is: 1H-NMR (DMSO-d6) δ(ppm) 7.3 (1H), 6.7 (2H), 8.0 (2H).

### Example 9: Synthesis of Compound ND401 2

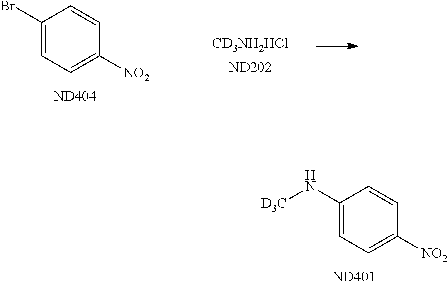

Compound ND402 was replaced by compound ND404, and the reaction was carried out according to the method of “Example 8: Synthesis of Compound ND401 1” to obtain compound ND401. The hydrogen nuclear magnetic resonance spectrum of compound ND401 is: 1H-NMR (DMSO-d6) δ (ppm) 7.3 (1H), 6.7 (2H), 8.0 (2H).

### Example 10: Synthesis of Compound ND401

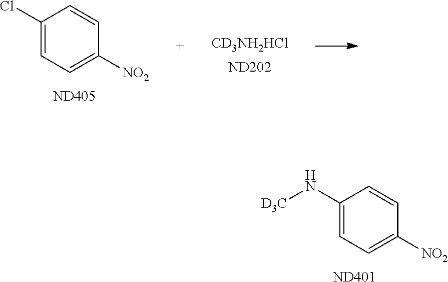

Compound ND402 was replaced by compound ND405, and the reaction was carried out according to the method of “Example 8: Synthesis of Compound ND401 1” to obtain compound ND401. The hydrogen nuclear magnetic resonance spectrum of compound ND401 is: 1H-NMR (DMSO-d6) δ (ppm) 7.3 (1H), 6.7 (2H), 8.0 (2H).

### Example 11: Synthesis of Compound ND401

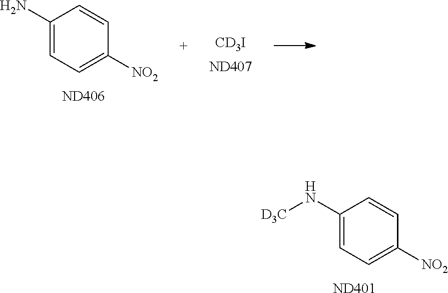

### Step 13: Synthesis of Compound ND401

p-Nitroaniline (compound ND406) (25 mmol) and trideuterated iodomethane (compound ND407) (10 mmol) were refluxed (65° C.) in tetradeuterated methanol (25 mL) for 10 hours. The reaction solution was cooled to room temperature and adjusted to alkalinity with 20% (w/v) potassium hydroxide. An aqueous solution (1.5 ml) of zinc chloride (1.5 g, 11 mmol) was added. The reaction mixture was cooled to 5° C., stirred and filtered, and the thick paste was extracted three times (3×30 mL) with petroleum ether (bp 60-80° C.). The organic extracts were combined and washed with water (3×30 mL) and 25% ammonia solution (30 mL). The organic layer was dried over anhydrous magnesium sulfate, filtered, and the filtrate was concentrated under reduced pressure and recrystallized with methanol to obtain compound ND401. The hydrogen nuclear magnetic resonance spectrum of compound ND401 is: 1H-NMR (DMSO-d6) δ (ppm) 7.3 (1H), 6.7 (2H), 8.0 (2H).

### Example 12: Synthesis of Compound ND801

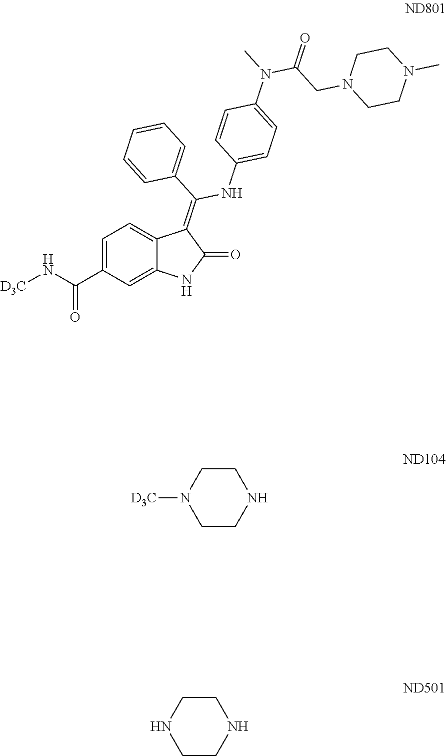 [SMILES: [2H]C([2H])([2H])NC(=O)c1ccc2c(c1)NC(=O)/C2=C(\Nc1ccc(N(C)C(=O)CN2CCN(C)CC2)cc1)c1ccccc1 ; [2H]C([2H])([2H])N1CCNCC1 ; C1CNCCN1]

Compound ND501 was used to replace compound ND104, and the reaction was carried out according to the method of “Example 1: Synthesis of Compound ND803” to obtain compound ND801. The hydrogen nuclear magnetic resonance spectrum of compound ND801 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (11H).

### Example 13: Synthesis of Compound ND802

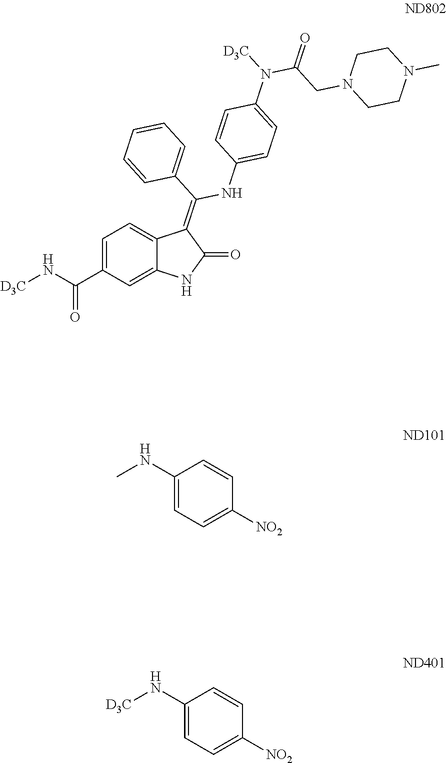

Compound ND401 was used to replace compound ND101, and the reaction was carried out according to the method of “Example 12: Synthesis of Compound ND801” to obtain compound ND802. The hydrogen nuclear magnetic resonance spectrum of compound ND802 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 2.7 (2H), 1.9-2.3 (11H).

### Example 14: Synthesis of Compound ND804

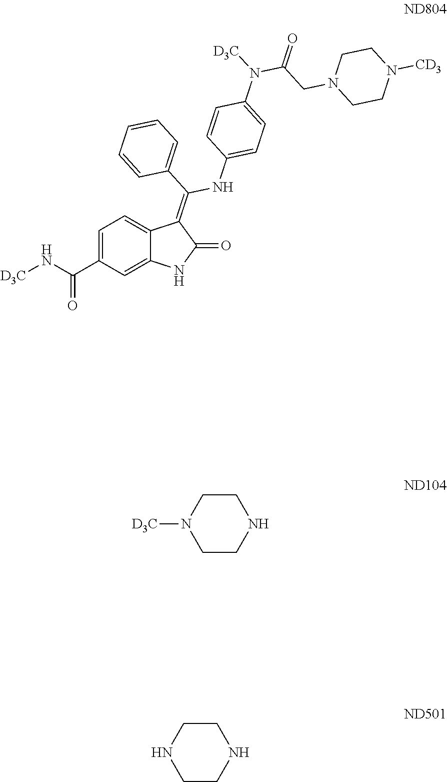 [SMILES: [2H]C([2H])([2H])NC(=O)c1ccc2c(c1)NC(=O)/C2=C(\Nc1ccc(N(C(=O)CN2CCN(C([2H])([2H])[2H])CC2)C([2H])([2H])[2H])cc1)c1ccccc1 ; [2H]C([2H])([2H])N1CCNCC1 ; C1CNCCN1]

Compound ND501 was replaced by compound ND104, and the reaction was carried out according to the method of “Example 13: Synthesis of Compound ND802” to obtain compound ND804. The hydrogen nuclear magnetic resonance spectrum of compound ND804 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 2.7 (2H), 1.9-2.3 (8H).

### Example 15: Synthesis of Compound ND805

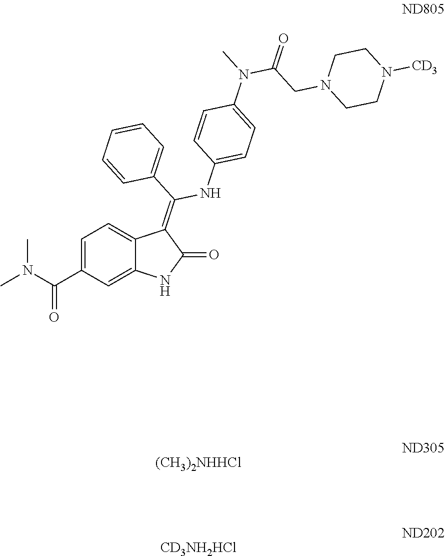 [SMILES: [2H]C([2H])([2H])N1CCN(CC(=O)N(C)c2ccc(N/C(=C3\C(=O)Nc4cc(C(=O)N(C)C)ccc43)c3ccccc3)cc2)CC1 ; "(CH3)2NHHCl" ; "CD3NH2HCl"]

Dimethylamine hydrochloride (compound ND305) was used to replace trideuterated methylamine hydrochloride (compound ND202), and the reaction was carried out according to the method of “Example 1: Synthesis of compound ND803” to obtain compound ND805. The hydrogen nuclear magnetic resonance spectrum of compound ND805 is: 1H-NMR (DMSO-d6) δ 12.1 (1H), 11.0 (1H), 7.4-7.6 (5H), 7.3 (1H), 6.8-7.2 (5H), 5.8 (1H), 3.1 (3H), 2.7-2.8 (8H), 1.9-2.3 (8H).

### Example 16: Synthesis of Compound ND806

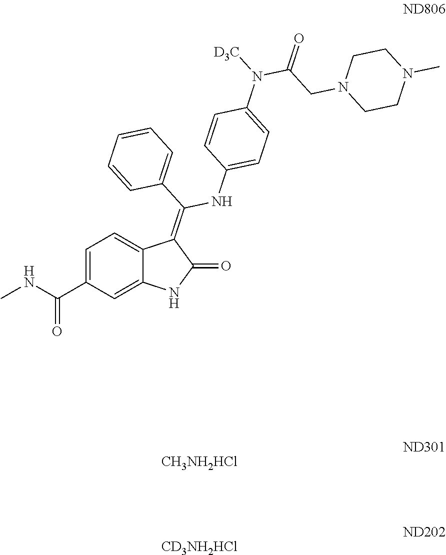

Methylamine hydrochloride (compound ND301) was used to replace trideuterated methylamine hydrochloride (compound ND202), and the reaction was carried out according to the method of “Example 13: Synthesis of compound ND802” to obtain compound ND806. The hydrogen nuclear magnetic resonance spectrum of compound ND806 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 2.7-2.8 (5H), 1.9-2.3 (11H).

### Example 17: Synthesis of Compound ND807

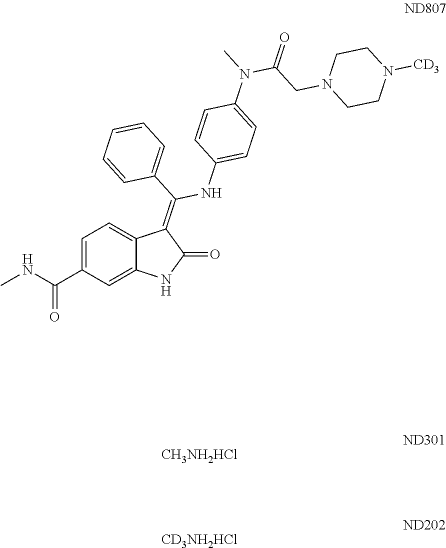 [SMILES: [2H]C([2H])([2H])N1CCN(CC(=O)N(C)c2ccc(N/C(=C3\C(=O)Nc4cc(C(=O)NC)ccc43)c3ccccc3)cc2)CC1 ; "CH3NH2HCl" ; "CD3NH2HCl"]

Methylamine hydrochloride (compound ND301) was used to replace trideuterated methylamine hydrochloride (compound ND202), and the reaction was carried out according to the method of “Example 1: Synthesis of compound ND803” to obtain compound ND807. The hydrogen nuclear magnetic resonance spectrum of compound ND807 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7-2.8 (5H), 1.9-2.3 (8H).

### Example 18: Synthesis of Compound ND808

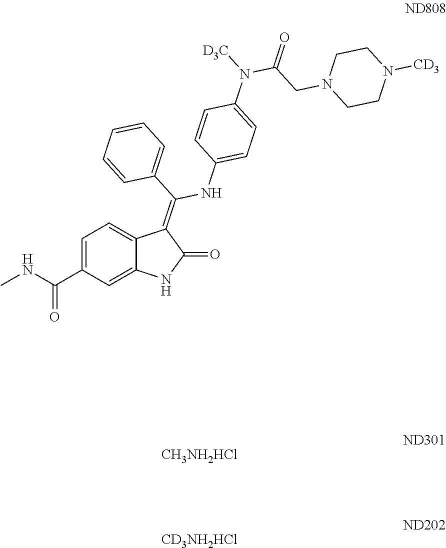 [SMILES: [2H]C([2H])([2H])N1CCN(CC(=O)N(c2ccc(N/C(=C3\C(=O)Nc4cc(C(=O)NC)ccc43)c3ccccc3)cc2)C([2H])([2H])[2H])CC1 ; "CH3NH2HCl" ; "CD3NH2HCl"]

Methylamine hydrochloride (compound ND301) was used to replace trideuterated methylamine hydrochloride (compound ND202), and the reaction was carried out according to the method of “Example 14: Synthesis of compound ND804” to obtain compound ND808. The hydrogen nuclear magnetic resonance spectrum of compound ND808 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 2.7-2.8 (5H), 1.9-2.3 (8H).

### Example 19: Synthesis of Compound ND809

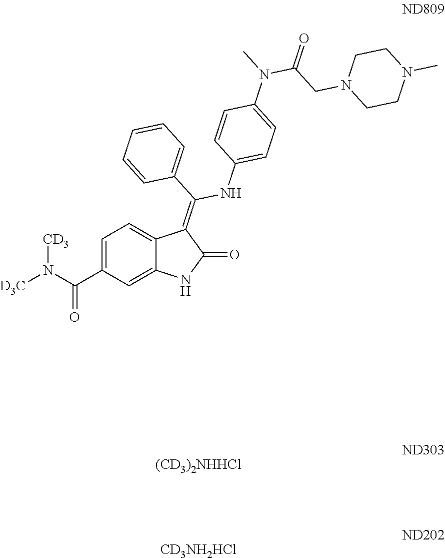 [SMILES: [2H]C([2H])([2H])N(C(=O)c1ccc2c(c1)NC(=O)/C2=C(\Nc1ccc(N(C)C(=O)CN2CCN(C)CC2)cc1)c1ccccc1)C([2H])([2H])[2H] ; "(CD3)2NHHCl" ; "CD3NH2HCl"]

The trideuterated methylamine hydrochloride (compound ND202) was replaced by hexadeuterated dimethylamine hydrochloride (compound ND303), and the reaction was carried out according to the method of “Example 12: Synthesis of compound ND801” to obtain compound ND809. The hydrogen nuclear magnetic resonance spectrum of compound ND809 is: 1H-NMR (DMSO-d6) δ 12.1 (1H), 11.0 (1H), 7.4-7.6 (5H), 7.3 (1H), 6.8-7.2 (5H), 5.8 (1H), 3.1 (3H), 2.7 (2H), 1.9-2.3 (11H).

### Example 20: Synthesis of Compound ND810

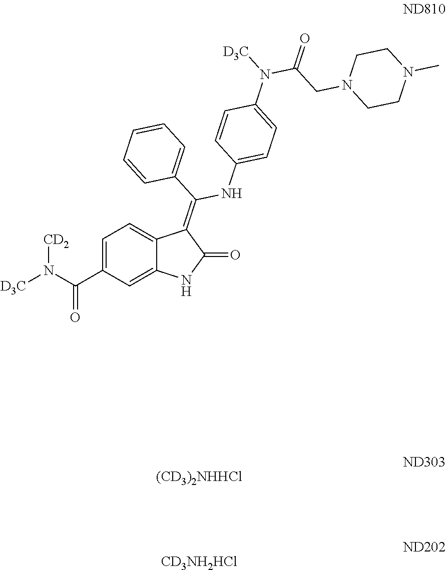

The trideuterated methylamine hydrochloride (compound ND202) was replaced by hexadeuterated dimethylamine hydrochloride (compound ND303), and the reaction was carried out according to the method of “Example 13: Synthesis of compound ND802” to obtain compound ND810. The hydrogen nuclear magnetic resonance spectrum of compound ND810 is: 1H-NMR (DMSO-d6) δ 12.1 (1H), 11.0 (1H), 7.4-7.6 (5H), 7.3 (1H), 6.8-7.2 (5H), 5.8 (1H), 2.7 (2H), 1.9-2.3 (11H).

### Example 21: Synthesis of Compound ND811

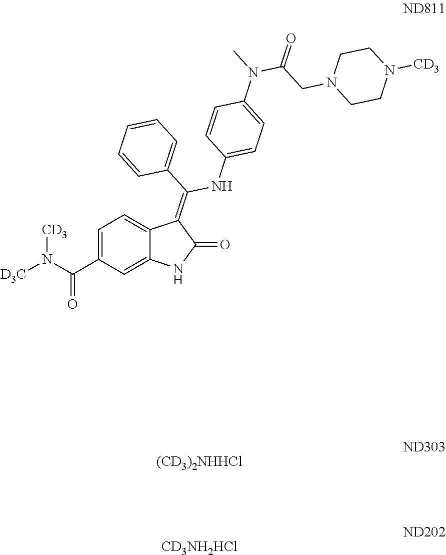 [SMILES: [2H]C([2H])([2H])N1CCN(CC(=O)N(C)c2ccc(N/C(=C3\C(=O)Nc4cc(C(=O)N(C([2H])([2H])[2H])C([2H])([2H])[2H])ccc43)c3ccccc3)cc2)CC1 ; "(CD3)2NHHCl" ; "CD3NH2HCl"]

Substituting hexadeuterated dimethylamine hydrochloride (compound ND303) for trideuterated methylamine hydrochloride (compound ND202), the reaction was carried out according to the method of “Example 1: Synthesis of compound ND803” to obtain compound ND811. The hydrogen nuclear magnetic resonance spectrum of compound ND811 is: 1H-NMVR (DMSO-d6) δ 12.1 (1H), 11.0 (1H), 7.4-7.6 (5H), 7.3 (1H), 6.8-7.2 (5H), 5.8 (1H), 3.1 (3H), 2.7 (2H), 1.9-2.3 (8H).

### Example 22: Synthesis of Compound ND812

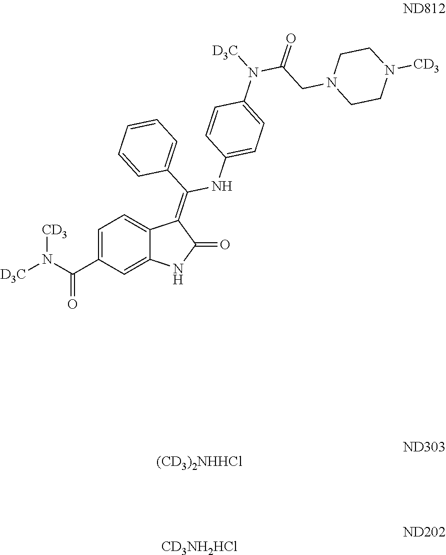 [SMILES: [2H]C([2H])([2H])N1CCN(CC(=O)N(c2ccc(N/C(=C3\C(=O)Nc4cc(C(=O)N(C([2H])([2H])[2H])C([2H])([2H])[2H])ccc43)c3ccccc3)cc2)C([2H])([2H])[2H])CC1 ; "(CD3)2NHHCl" ; "CD3NH2HCl"]

The trideuterated methylamine hydrochloride (compound ND202) was replaced by hexadeuterated dimethylamine hydrochloride (compound ND303), and the reaction was carried out according to the method of “Example 14: Synthesis of compound ND804” to obtain compound ND812. The hydrogen nuclear magnetic resonance spectrum of compound ND812 is: 1H-NMR (DMSO-d6) δ 12.1 (1H), 11.0 (1H), 7.4-7.6 (5H), 7.3 (1H), 6.8-7.2 (5H), 5.8 (1H), 2.7 (2H), 1.9-2.3 (8H).

### Example 23: Synthesis of Compound ND813

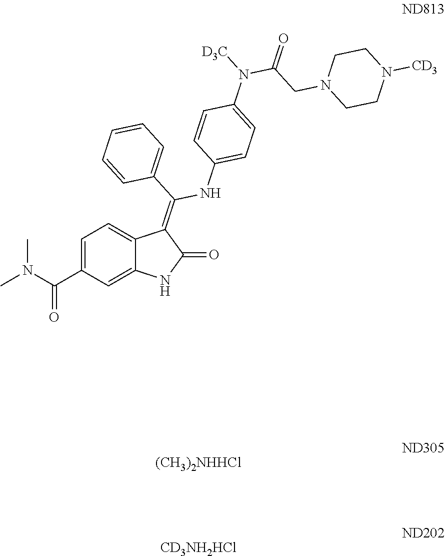 [SMILES: [2H]C([2H])([2H])N1CCN(CC(=O)N(c2ccc(N/C(=C3\C(=O)Nc4cc(C(=O)N(C)C)ccc43)c3ccccc3)cc2)C([2H])([2H])[2H])CC1 ; "(CH3)2NHHCl" ; "CD3NH2HCl"]

Dimethylamine hydrochloride (compound ND305) was used to replace trideuterated methylamine hydrochloride (compound ND202), and the reaction was carried out according to the method of “Example 14: Synthesis of compound ND804” to obtain compound ND813. The hydrogen nuclear magnetic resonance spectrum of compound ND813 is: 1H-NMR (DMSO-d6) δ 12.1 (1H), 11.0 (1H), 7.4-7.6 (5H), 7.3 (1H), 6.8-7.2 (5H), 5.8 (1H), 2.7-2.8 (8H), 1.9-2.3 (8H).

### Example 24: Synthesis of Compound ND814

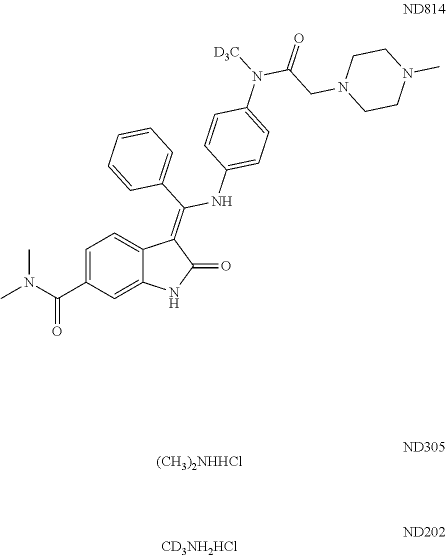

Dimethylamine hydrochloride (compound ND305 was use to replace trideuterated methylamine hydrochloride (compound ND202), and the reaction was carried out according to the method of “Example 13: Synthesis of compound ND802” to obtain compound ND814. The nuclear magnetic resonance hydrogen spectrum of compound ND814 is: 1H-NMR (DMSO-d6) δ 12.1 (1H), 11.0 (1H), 7.4-7.6 (5H), 7.3 (1H), 6.8-7.2 (5H), 5.8 (1H), 2.7-2.8 (8H), 1.9-2.3 (11H).

### Example 25: Synthesis Method 2 of Compound ND809

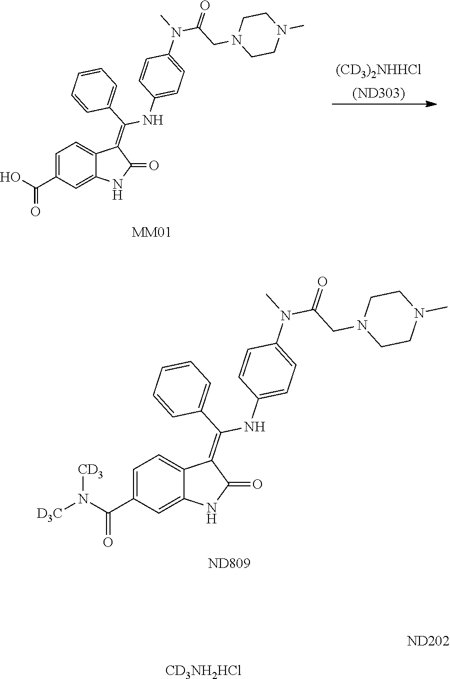

The trideuterated methylamine hydrochloride (compound ND202) was replaced by hexadeuterated methylamine hydrochloride (compound ND303), and the reaction was carried out according to the method of “Example 3: Synthesis Method 2 of Compound ND801” to obtain compound ND809. The hydrogen nuclear magnetic resonance spectrum of compound ND809 is: 1H-NMR (DMSO-d6) δ 12.1 (1H), 11.0 (1H), 7.4-7.6 (5H), 7.3 (1H), 6.8-7.2 (5H), 5.8 (1H), 3.1 (3H), 2.7 (2H), 1.9-2.3 (11H).

### Example 26: Synthesis of Compound ND322

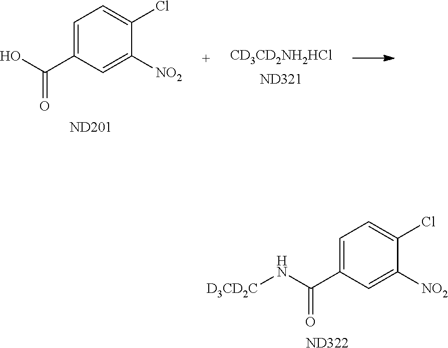

Compound ND321 was used to replace compound ND301, and the reaction was carried out according to the method of “Example 4: Synthesis of compound ND302” to obtain compound ND322.

### Example 27: Synthesis of Compound ND324

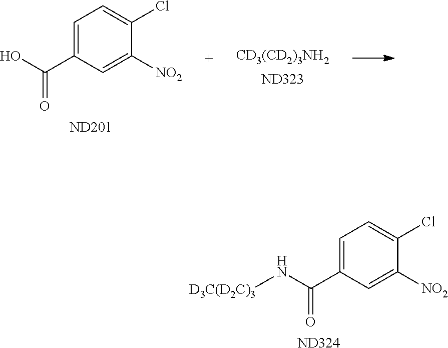

Compound ND323 was used to replace compound ND301, and the reaction was carried out according to the method of “Example 4: Synthesis of compound ND302” to obtain compound ND324.

### Example 28: Synthesis of Compound ND815

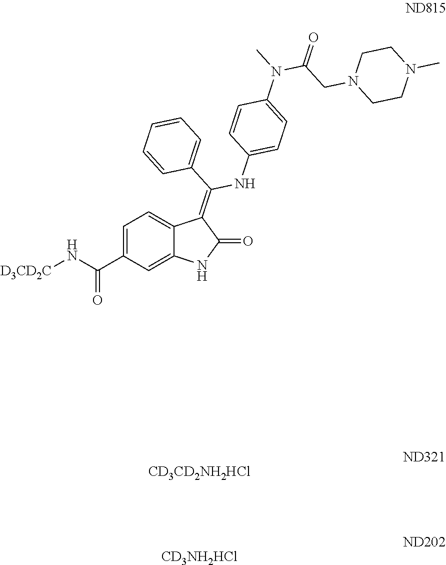

Ethyl-d5-amine hydrochloride (compound ND321) was used to replace trideuterated methylamine hydrochloride (compound ND202), and the reaction was carried out according to the method of “Example 12: Synthesis of compound ND801” to obtain compound ND815. The hydrogen nuclear magnetic resonance spectrum of compound ND815 is: 1H-NMR (DMSO-d6) 612.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (11H).

### Example 29: Synthesis of Compound ND815 2

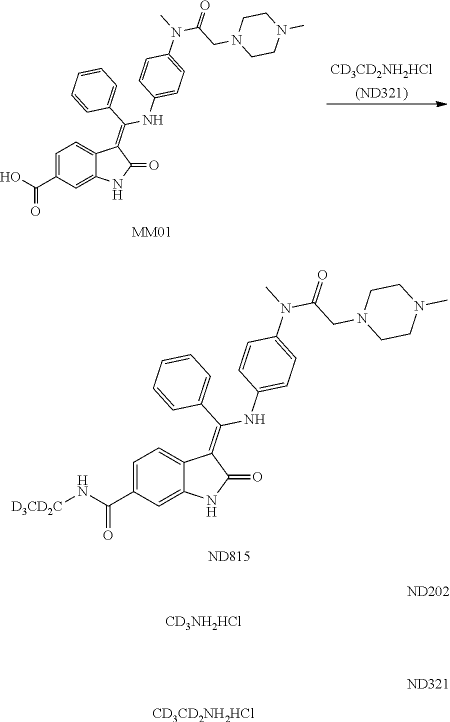

Ethyl-d5-amine hydrochloride (compound ND321) was used to replace trideuterated methylamine hydrochloride (compound ND202), and the reaction was carried out according to the method of “Example 3: Synthesis Method 2 of Compound ND801” to obtain compound ND815. The hydrogen nuclear magnetic resonance spectrum of compound ND815 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (11H).

### Example 30: Synthesis of Compound ND816

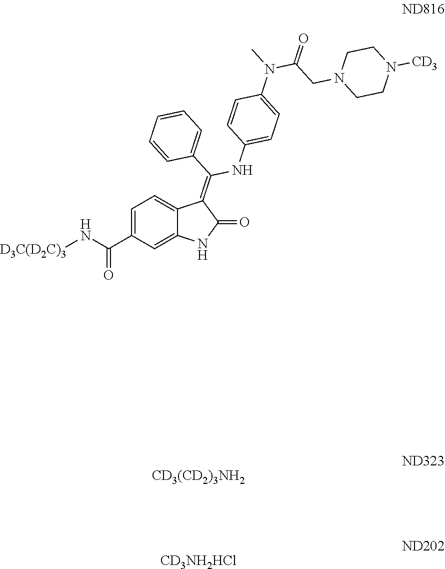

Trideuterated methylamine hydrochloride (compound ND202) was replaced with n-butyl-d9-amine (compound ND323), and the reaction was carried out according to the method of “Example 1: Synthesis of compound ND803” to obtain compound ND816. The hydrogen nuclear magnetic resonance spectrum of compound ND816 is: 1H-NMR (DMSO-d6) δ 12.1 (1H), 11.0 (1H), 8.2 (1H), 7.4-7.6 (5H), 7.3 (1H), 6.8-7.2 (5H), 5.8 (1H), 3.1 (3H), 2.7 (2H), 1.9-2.3 (8H).

### Example 31: In Vitro Kinase Inhibition Assay

The pathogenesis of fibrosis is still unclear. The current view is that blocking the intracellular signal transduction pathway of VEGFR, FGFR and/or PDGFR can inhibit the proliferation, migration and transformation of fibroblasts into myofibroblasts, thereby reducing the degree of fibrosis. Therefore, it is believed that the inhibition of kinases FGFR, VEGFR and/or PDGFR can improve pulmonary fibrosis. This example measures the inhibitory activity of the compounds of the present invention against kinases FGFR-1, VEGFR2 and PDGFRα.

The inhibitory activity of the compounds against kinases FGFR-1, VEGFR2 and PDGFRα was detected using the mobility shift assay method at Km ATP (Km ATP represents the ATP concentration corresponding to half the maximum reaction rate of kinase and ATP).

Corning 3674 white 384-well assay plate, kinase VEGFR2 (Invitrogen), kinase FGFR-1 (Invitrogen), kinase PDGFRα (Invitrogen), ATP (Sigma) were used. The buffer solution included 50 mM HEPES (pH 7.5), 0.015% (v/v) Brij-35, 10 mM MgCl2, and 2 mM DTT. The stop solution included 100 mM HEPES (pH 7.5), 0.015% (v/v) Brij-35, 0.2% (v/v) Coating Reagent #3, and 50 mM EDTA. The enzyme solution was obtained by taking a kinase with a concentration (unit is the titer of the kinase) of 2.5 times and adding it to 1 volume of buffer. The substrate solution was obtained by taking a substrate “FAM-labeled polypeptide and ATP” with a concentration corresponding to the kinase with a concentration (unit is the titer of the kinase) of 2.5 times and adding it to 1 volume of buffer.

The compound of this example was diluted with DMSO to a 500 M solution, and then diluted three times with DMSO to a minimum concentration of 250 nM, for a total of 10 concentrations. Take 10 μL of each of the 10 concentrations of the compound, add 90 μL of buffer, and the 10 concentrations of the compound to be tested are obtained. Take 5 μL of each of the 10 concentrations of the compound to be tested, add 10 μL of enzyme solution, and incubate at room temperature for 10 minutes; then add 10 μL of substrate solution, incubate at 28° C. for an appropriate time (the time is adjusted for different kinases); then add 25 μL of stop solution to terminate the reaction. Read the value.

The inhibition percentage was calculated according to the following formula:

$$Inhibition \% = [ 1 - ( {A}_{Compound} - {A}_{min} ) / ( {A}_{max} - {A}_{min} ) ] \times 100$$

Wherein, A_{compound} is the reading at the concentration to be tested of the compound of this example, Aₘᵢₙ is the reading without adding kinase, and Amax is the reading without adding the compound of this example. The IC₅₀ values of each compound are calculated and shown in Table 1 below.

TABLE 1
IC₅₀ values of the compounds of this example against
kinases VEGFR2, FGFR-1 and PDGFRα (unit: nM)

| Compound | VEGFR2 | FGFR-1 | PDGFRα |
|---|---|---|---|
| Example compound | W1 | W1 | W2 |

Marking (grade): W1 is 10-3000; W2 is 50-3000.

The results show that the IC₅₀ values of the compounds of this example for VEGFR2 and FGFR-1 are all between 10-3000 nM; the IC₅₀ values for PDGFRα are all between 50-3000 nM, indicating that the compounds of this example have inhibitory effects on kinases VEGFR2, FGFR-1 and PDGFRα, and have the potential to improve pulmonary fibrosis.

### Example 32: Pharmacokinetics in Rats

Forty-two male Sprague-Dawley rats, 6-9 weeks old, weighing about 220 g, were divided into 7 groups (compound ND801 group, compound ND802 group, compound ND803 group, compound ND805 group, compound ND807 group, compound ND815 group and nintedanib group), 6 rats in each group. According to the grouping, a single oral administration of 30 mg/kg dose of compound ND801, compound ND802, compound ND803, compound ND805, compound ND807, compound ND815 or nintedanib was performed to compare the pharmacokinetic differences. Rats were fasted 12 hours before administration. The dosing solution was prepared with 0.5% sodium carboxymethylcellulose (CMC-Na). Blood was collected from the eye sockets at 0.25 hours, 0.5 hours, 1 hour, 2 hours, 4 hours, 6 hours, 8 hours, 12 hours, 24 hours and 48 hours after administration. After the plasma was separated from the blood sample, it was stored in a −80° C. refrigerator for future use. An LC-MS/MS analysis method was established to measure the plasma samples.

The test results show that compared with nintedanib, the relative bioavailability and/or elimination half-life T1/2 and/or area under the curve AUC and/or maximum blood concentration Cmax of compound ND801, compound ND802, compound ND803, compound ND805, compound ND807 or compound ND815 increased by more than 100%.

From the present results, it can be seen that compared with nintedanib, compound ND801, compound ND802, compound ND803, compound ND805, compound ND807 and/or compound ND815 of the present invention have better pharmacokinetic properties in rats, indicating better pharmacodynamics and therapeutic effects.

### Example 33: Pharmacokinetics in Rats

Forty-two male Sprague-Dawley rats, 6-9 weeks old, weighing about 220 g, were divided into 7 groups (compound ND809 group, compound ND810 group, compound ND812 group, compound ND813 group, compound ND814 group, compound ND816 group and nintedanib group), 6 rats in each group. According to the grouping, a single oral administration of 30 mg/kg dose of compound ND809, compound ND810, compound ND812, compound ND813, compound ND814, compound ND816 or nintedanib was given to compare their pharmacokinetic differences. Rats were fasted 12 hours before administration. The dosing solution was prepared with 0.5% sodium carboxymethylcellulose (CMC-Na). Blood was collected from the eye sockets at 0.25 hours, 0.5 hours, 1 hour, 2 hours, 4 hours, 6 hours, 8 hours, 12 hours, 24 hours and 48 hours after administration. After the plasma was separated from the blood sample, it was stored in a −80° C. refrigerator for future use. An LC-MS/MS analysis method was established to measure the plasma samples.

The test results show that compared with nintedanib, the relative bioavailability and/or elimination half-life T1/2 and/or area under the curve AUC and/or maximum blood concentration Cmax of compound ND809, compound ND810, compound ND812, compound ND813, compound ND814 or compound ND816 increased by more than 100%.

From the present results, it can be seen that compared with nintedanib, compound ND809, compound ND810, compound ND812, compound ND813, compound ND814 and/or compound ND816 of the present invention have better pharmacokinetic properties in rats, indicating better pharmacodynamics and therapeutic effects.

### Example 34: Inhibition of Bleomycin-Induced Pulmonary Fibrosis in Mice

The bleomycin (BLM) model is a classic animal model of pulmonary fibrosis. It has become the most widely used animal model of pulmonary fibrosis because it meets many characteristics of pulmonary fibrosis and other fibrotic interstitial lung diseases (ILD), as well as good repeatability and inducibility. At present, the evaluation indicators of animal models of pulmonary fibrosis (PF) mainly include observation of the general state of the animal; fibrosis-related factors, such as hydroxyproline (HYP); lung function indicators, such as forced vital capacity (FVC), respiratory dynamic compliance (Cdyn), inspiratory airway resistance (Ri), expiratory airway resistance (Re); lung tissue pathological examination, such as fibrosis area.

In this example, bleomycin was injected into the trachea to establish a pulmonary fibrosis model in C57BL/6 male mice. After modeling, compared with the control group mice, the content of HYP in the lung tissue of the model group was significantly increased (P<0.01); compared with the control group mice, the FVC and Cdyn of the model group were significantly reduced (P<0.01), Ri was significantly increased (P<0.01), and Re was increased; the mice had obvious pulmonary fibrosis, and the fibrosis area was equal to or greater than 20%. In this example, a comparative study was conducted on the improvement of nintedanib (60 mg/kg) and the example compound (40 mg/kg) on mouse pulmonary fibrosis.

### (1) Experimental Animals

Forty SPF C57BL/6 male mice, 8-10 weeks old, weighing (25±2 g), were allowed free access to water and feed, and maintained under a day-night cycle at a temperature of 25±2° C. and a relative humidity of 50±10%.

### (2) Animal Grouping and Drug Administration

Forty male C57BL/6 mice were divided into four groups, 10 mice in each group, namely control group, model group, nintedanib and example compound group. On the first day, mice in the model group, nintedanib group and example compound group were modeled with bleomycin (2 mg/kg). The operation was as follows: the mice were anesthetized by intraperitoneal injection of 5% chloral hydrate, and the trachea was separated and exposed. A 1 mL injection needle was used to draw the bleomycin solution, and the mouse trachea was pierced between the two ring bones, and then the bleomycin solution was injected. After the administration, the mouse was upright and rotated left and right to evenly distribute the bleomycin solution in the lungs. Under the same conditions, the mice in the control group were instilled with an equal volume of normal saline in the trachea. On the 8th-14th day, the nintedanib group was gavaged with 60 mg/kg of nintedanib daily; the example compound group was gavaged with 40 mg/kg of the example compound; the control group and the model group were given equal volumes of normal saline.

The behavior, activity, food intake, urine, feces and other symptoms of the animals before and after administration were observed every day, and the body weight was weighed.

### (3) Data Processing

The experimental data were expressed as mean±standard deviation (±s) and analyzed using SPSS 22.0 statistical software. One-way analysis of variance was used to compare the differences between groups. p<0.05 indicated a statistically significant difference; p<0.01 indicated a significant difference.

### (4) Determination of Hydroxyproline (HYP) Content in Lung Tissue

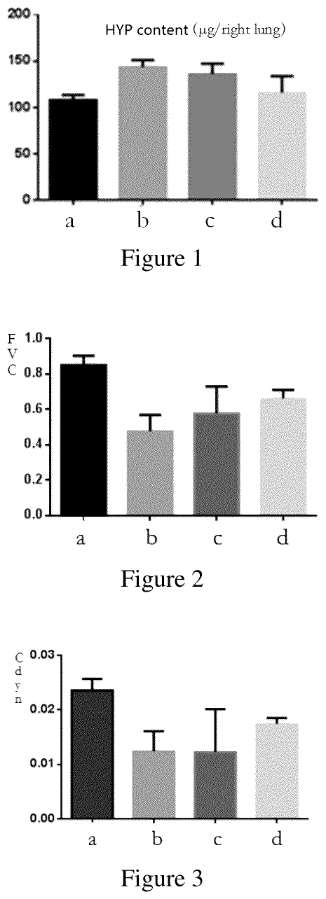

After the mice were killed on the 15th day, the right lungs of the mice in the control group, model group, nintedanib and example compound groups were minced and mixed, and then 9 times the volume of pre-cooled physiological saline was added and homogenized. The operation was performed according to the instructions of the hydroxyproline detection kit, and the hydroxyproline content of the right lung was calculated. The results of the determination of hydroxyproline (HYP) content in the right lung of mice are shown in FIG. 1 and Table 2:

TABLE 2
Comparison of hydroxyproline (HYP)
content in the right lung of mice

| | | | | Example |
|---|---|---|---|---|
| | Control | Model | Nintedanib | Compound |
| Group | Group | Group | Group | Group |
| HYP Average Value | A1 | A2 | A3 | A4 |
| (μg/right lung) | | | | |

Marking (grade): A1 is 100-116; A2 is 127-148; A3 is 124-143; A4 is 108-123.

### (5) Mouse Lung Function Test

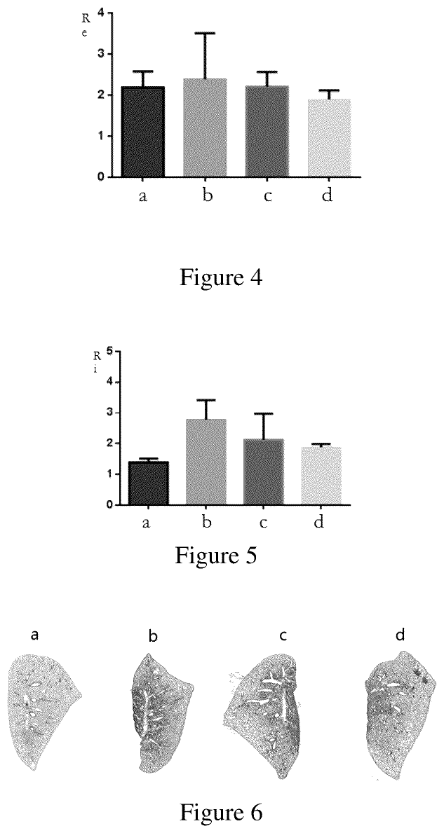

On the 15th day, the mice were anesthetized with 500 chloral hydrate, the trachea was separated layer by layer, and the trachea was intubated. The other end of the trachea was connected to the mouse body anchor box, and the body anchor box was sealed. The animal lung function analysis system in the pulmonary function instrument was used to collect forced vital capacity (FVC), respiratory dynamic compliance (Cdyn), expiratory airway resistance (Re), and inspiratory airway resistance (Ri). The results of mouse FVC are shown in FIG. 2 and Table 3; the results of mouse Cdyn are shown in FIG. 3 and Table 4; the results of mouse Re are shown in FIG. 4; and the results of mouse Ri are shown in FIG. 5.

TABLE 3
Results of mouse FVC determination

| | | | | Example |
|---|---|---|---|---|
| | Control | Model | Nintedanib | Compound |
| Group | Group | Group | Group | Group |
| FVC | B1 | B2 | B3 | B4 |
| Average | | | | |
| Value | | | | |

Marking (grade): B1 is 0.78-0.92; B2 is 0.33-0.51; B3 is 0.52-0.62; B4 is 0.62-0.72.

TABLE 4
Mouse Cdyn assay results

| | | | | Example |
|---|---|---|---|---|
| | Control | Model | Nintedanib | Compound |
| Group | Group | Group | Group | Group |
| Cdyn Average | C1 | C2 | C3 | C4 |
| Value | | | | |

Marking (grade): C1 is 0.020-0.024; C2 is 0.009-0.015; C3 is 0.010-0.016; C4 is 0.016-0.020.

### (6) Determination of Pulmonary Fibrosis Area

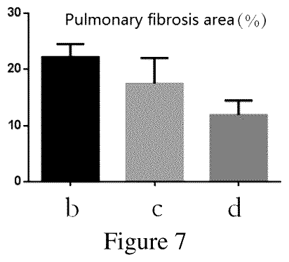

After the mice were killed on the 15th day, the lung tissues were dehydrated, embedded, sliced, and HE stained. The lung tissue pathology was observed under a light microscope, and the pulmonary fibrosis area was counted using Image Pro Plus software to calculate the percentage of pulmonary fibrosis area. The HE staining image is shown in FIG. 6; the percentage of pulmonary fibrosis area is shown in FIG. 7 and Table 5.

TABLE 5
Percentage of lung fibrosis area

| | | | Example |
|---|---|---|---|
| | Model | Nintedanib | Compound |
| Group | Group | Group | Group |
| Average Area of Pulmonary | D1 | D2 | D3 |
| Fibrosis (%) | | | |

Marking (grade): D1 is 20-27; D2 is 13-19; D3 is 7-13.

As can be seen from the above figures and tables, the results of the determination of hydroxyproline (HYP) in lung tissue show that the hydroxyproline content in the lung tissue of the model group was significantly higher than that in the control group; compared with the model group, the HYP content in the lung tissue of the nintedanib group was reduced; compared with the model group, the HYP content in the lung tissue of the example compound group was reduced; and the HYP content in the lung tissue of the example compound group was lower than that of the nintedanib group.

The results of mouse lung function test showed that the FVC and Cdyn of the model group were significantly lower than those of the control group; compared with the model group, the FVC of the nintedanib group increased and Cdyn did not change much; compared with the model group, the FVC and Cdyn of the example compound group increased significantly; and the FVC and Cdyn of the example compound group were significantly greater than those of the nintedanib group.

The results of the mouse lung function test showed that the Ri and Re of the Example compound group were improved compared with the model group; and the improvement effects of Ri and Re of the Example compound group were significantly better than those of the Nintedanib group. The results of the measurement of the pulmonary fibrosis area showed that compared with the model group, the pulmonary fibrosis area (%) of the Nintedanib group was significantly reduced; compared with the model group, the pulmonary fibrosis area (%) of the Example compound group was significantly reduced; and the pulmonary fibrosis area (%) of the Example compound group was significantly smaller than that of the Nintedanib group.

The above results all indicate that the therapeutic effect of the example compound (40 mg/kg) on the bleomycin-induced mouse pulmonary fibrosis model is significantly better than that of nintedanib (60 mg/kg).

### Example 35: Inhibition of Bleomycin-Induced Pulmonary Fibrosis in Mice

In this example, bleomycin was injected into the trachea to create a pulmonary fibrosis model in C57BL/6 male mice. The improvement effects of the compound in this example, compound ND901 and compound ND902 on the area (%) of pulmonary fibrosis were compared. The structural formulas of compound ND901 and compound ND902 are shown below:

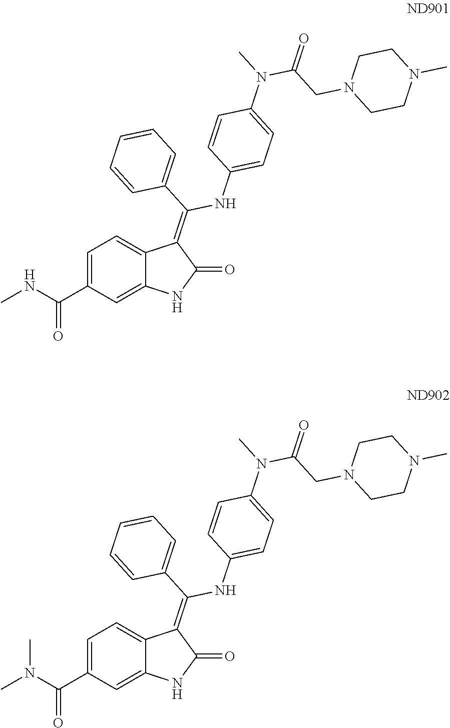

After bleomycin was injected into the trachea to create a pulmonary fibrosis model in C57BL/6 male mice, the mice had obvious pulmonary fibrosis, and the fibrosis area was equal to or greater than 20%. This example compared the improvement of the pulmonary fibrosis area (%) of the example compound (40 mg/kg), the compound ND901 (40 mg/kg) and the compound ND902 (40 mg/kg) on the mice.

### (1) Experimental Animals

Twenty-five SPF C57BL/6 male mice, 8-10 weeks old, weighing (25±2 g), were allowed free access to water and feed, and maintained under a day-night cycle at a temperature of 25±2° C. and a relative humidity of 50±10%.

### (2) Animal Grouping and Drug Administration

Twenty-five male C57BL/6 mice were divided into five groups, 5 mice in each group, namely control group, model group, example compound group, compound ND901 group and compound ND902 group. On the first day, mice in the model group, example compound group, compound ND901 group and compound ND902 group were modeled with bleomycin (2 mg/kg). The operation was as follows: the mice were anesthetized by intraperitoneal injection of 5% chloral hydrate, and the trachea was separated and exposed. Use a 1 mL injection needle to draw the bleomycin solution, pierce the mouse trachea between the two ring bones, and then inject the bleomycin solution. After the administration, the mice were upright and rotated left and right to evenly distribute the bleomycin solution in the lungs. Under the same conditions, the control group mice were instilled with an equal volume of normal saline in the trachea. On days 8-14, the Example compound group was gavaged with 40 mg/kg of the Example compound daily; the Compound ND901 group was gavaged with 40 mg/kg of the Compound ND901; the Compound ND902 group was gavaged with 40 mg/kg of the Compound ND902; the control group and the model group were respectively given an equal volume of physiological saline. The behavior, activity, food intake, urine, feces and other symptoms of the animals before and after administration were observed every day, and the body weight was measured.

### (3) Data Processing

The experimental data were expressed as mean±standard deviation (±s) and analyzed using SPSS 22.0 statistical software. One-way analysis of variance was used to compare the differences between groups. p<0.05 indicated a statistically significant difference; p<0.01 indicated a significant difference.

### (4) Determination of Pulmonary Fibrosis Area

After the mice were killed on the 15th day, the lung tissues were dehydrated, embedded, sliced, and HE stained. The lung tissue pathology was observed under a light microscope, and the pulmonary fibrosis area was counted using Image Pro Plus software to calculate the percentage of pulmonary fibrosis area. The percentage of pulmonary fibrosis area is shown in Table 6.

TABLE 6
Percentage of lung fibrosis area

| | Average Area of |
|---|---|
| Group | Pulmonary Fibrosis (%) |
| Model Group | E1 |
| Example Compound | E2 |
| Compound ND901 | E3 |
| Compound ND902 | E3 |

Marking (grade): E1 is 20-28; E2 is 6-13; E3 is 14-20.

The results of the measurement of pulmonary fibrosis area showed that compared with the model group, the pulmonary fibrosis area (%) of the compound ND901 group and the compound ND902 group was reduced; compared with the model group, the pulmonary fibrosis area (%) of the example compound group was significantly reduced; and the pulmonary fibrosis area (%) of the example compound group was significantly smaller than that of the compound ND901 group and the compound ND902 group.

This result shows that the therapeutic effect of the example compound (40 mg/kg) on the bleomycin-induced mouse pulmonary fibrosis model is significantly better than that of compound ND901 (40 mg/kg) and compound ND902 (40 mg/kg).

### Example 36: HERG Experiment

In this example, hERG experiments were used to predict the cardiac toxicity of the compound in this example. Cisapride was used as a positive control, and the IC₅₀ of hERG was measured to be 0.020 μM. This proves that this method can be used for comparative studies of hERG experiments between the compound in this example and Nintedanib. In this experiment, the IC₅₀ of hERG for Nintedanib was measured to be 4.9 μM. The IC₅₀ of the compound in this example is greater than 30 μM, indicating that the cardiac toxicity risk of the candidate compound is lower than that of Nintedanib.

### Example 37: Mouse Toxicity Experiment

This example compared the mouse toxicity of the compound in this example with compound NND05 (structural formula shown in the figure below).

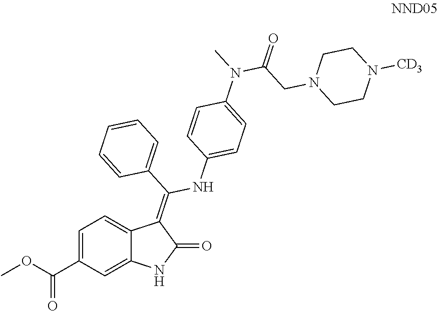 [SMILES: [2H]C([2H])([2H])N1CCN(CC(=O)N(C)c2ccc(N/C(=C3\C(=O)Nc4cc(C(=O)OC)ccc43)c3ccccc3)cc2)CC1]

24 mice (half male and half female) were randomly divided into 3 groups (solvent group, compound group of this example, and compound NND05 group), with 8 mice in each group (half male and half female). The mice were orally administered with solvent, compound of this example, and compound NND05 at a dose of 800 mg/kg once a day for one week. The body weight changes of mice in the solvent group, compound group, and compound NND05 group were measured to be 115.3±5.8(%), 103.4±5.4(%), and 92.9±12.5(%), respectively. Liver slices of mice showed no liver damage in the solvent group, while both compound and compound NND05 groups had liver damage, with compound NND05 group showing more severe liver damage than compound group in this example. And no mice died in the solvent group, 2 mice (both male) died in the compound group of this example, and 5 mice (3 male and 2 female) died in the compound NND05 group. This indicates that the toxicity (such as hepatotoxicity) of compound NND05 in this experiment is greater than that of the compound in this example.

Finally, it is necessary to note that the above detailed description of specific embodiments of the invention is only for illustration, and the invention is not limited to the specific embodiments described above. For those skilled in the art, any equivalent modifications and substitutions to the invention are also within the scope of the invention. Therefore, all equivalent transformations and modifications made without departing from the spirit and scope of the invention should be included within the scope of the invention.

1. A compound represented by formula I or a pharmaceutically acceptable salt thereof: 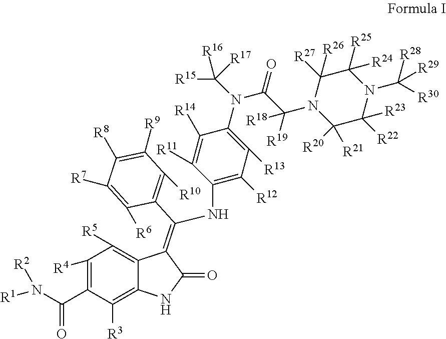
wherein:
R¹ and R² are each independently selected from H, deuterium (D), C₁₋₄ alkyl, C₂₋₄ alkenyl, C₂₋₄ alkynyl, 3-6-membered saturated or unsaturated carbocyclic ring, 3-6-membered saturated or unsaturated heterocyclic ring containing nitrogen (N) and/or oxygen (O) and/or sulphur (S), wherein the C₁₋₄ alkyl, C₂₋₄ alkenyl, C₂₋₄ alkynyl, 3-6-membered saturated or unsaturated carbocyclic ring, 3-6-membered saturated or unsaturated heterocyclic ring containing nitrogen (N) and/or oxygen (O) and/or sulphur (S) are optionally substituted by zero or more substituents selected from deuterium, halogen, cyano, hydroxyl, sulfhydryl; or R¹ and R², together with the atoms to which they are attached, form a ring;
R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are each independently selected from hydrogen (H) or deuterium (D);
the conditions are R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are all hydrogen, the R¹ or R² group contains at least one deuterium atom.

2. The compound according to claim 1, wherein the compound is one of the compounds shown below:

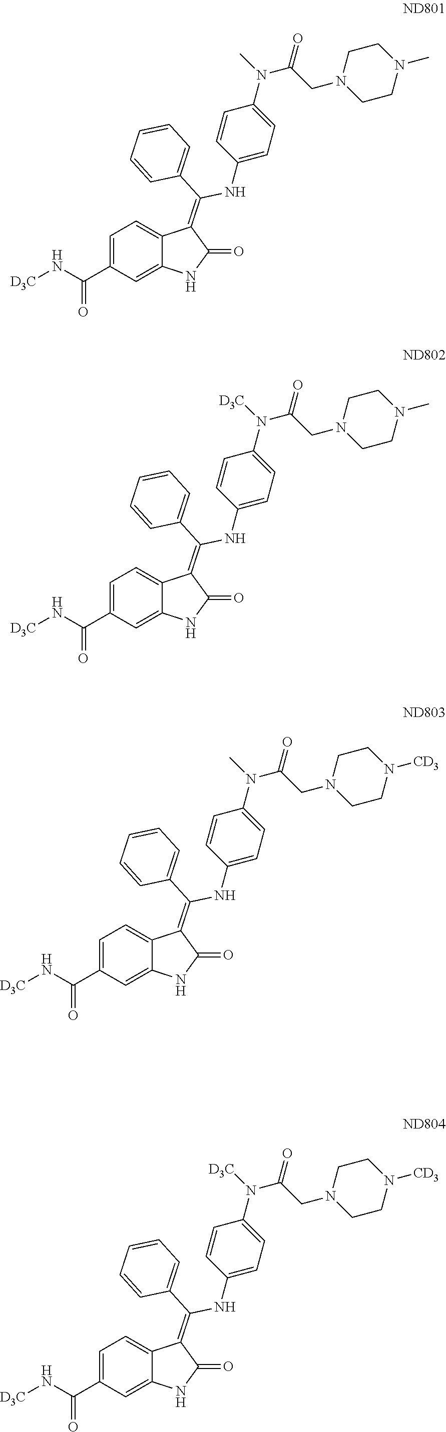 [SMILES: [2H]C([2H])([2H])NC(=O)c1ccc2c(c1)NC(=O)/C2=C(\Nc1ccc(N(C)C(=O)CN2CCN(C)CC2)cc1)c1ccccc1 ; [2H]C([2H])([2H])NC(=O)c1ccc2c(c1)NC(=O)/C2=C(\Nc1ccc(N(C(=O)CN2CCN(C)CC2)C([2H])([2H])[2H])cc1)c1ccccc1 ; [2H]C([2H])([2H])NC(=O)c1ccc2c(c1)NC(=O)/C2=C(\Nc1ccc(N(C)C(=O)CN2CCN(C([2H])([2H])[2H])CC2)cc1)c1ccccc1 ; [2H]C([2H])([2H])NC(=O)c1ccc2c(c1)NC(=O)/C2=C(\Nc1ccc(N(C(=O)CN2CCN(C([2H])([2H])[2H])CC2)C([2H])([2H])[2H])cc1)c1ccccc1]

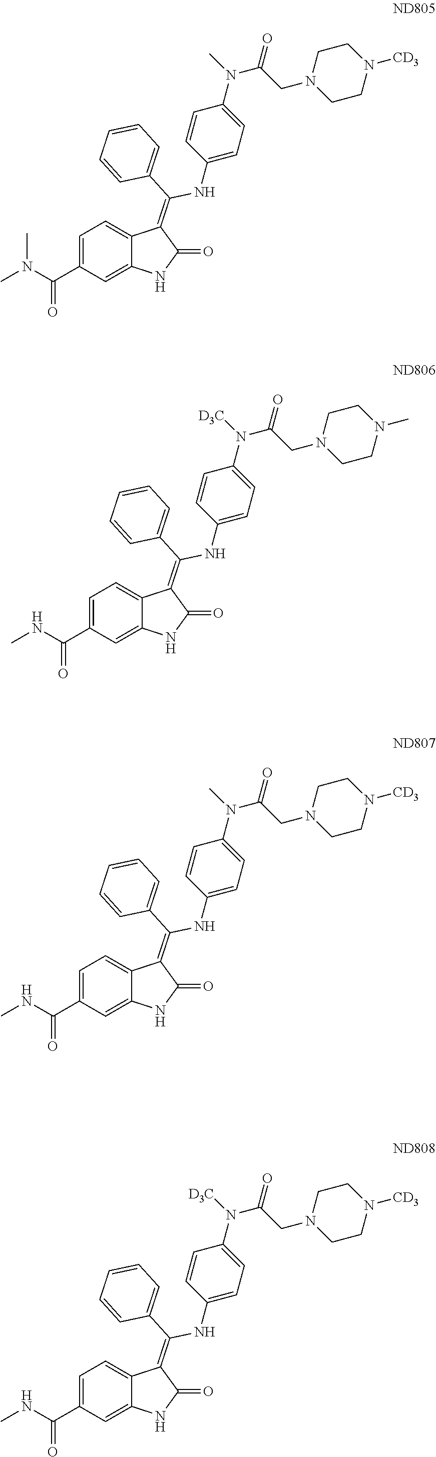

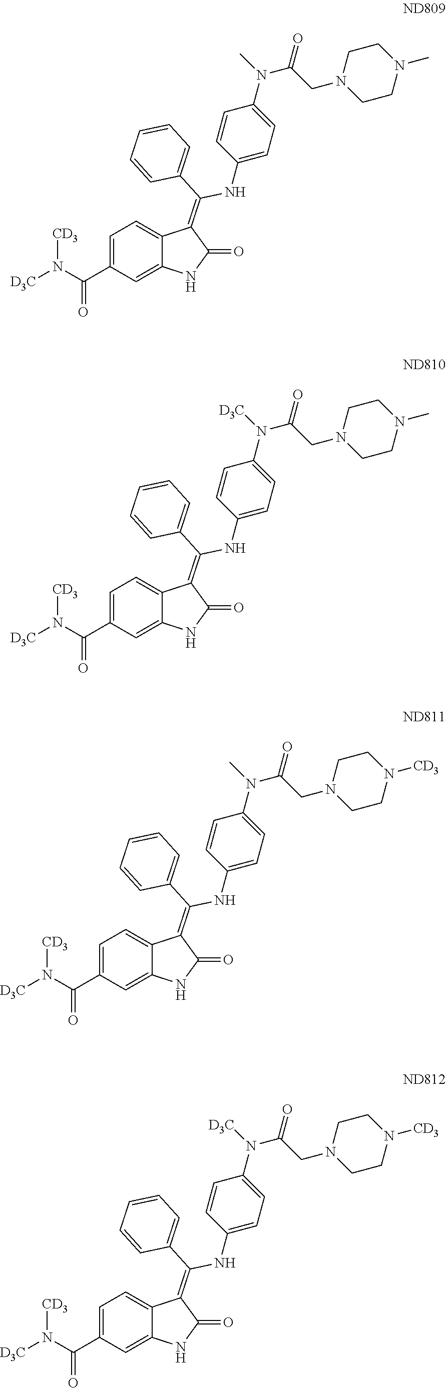

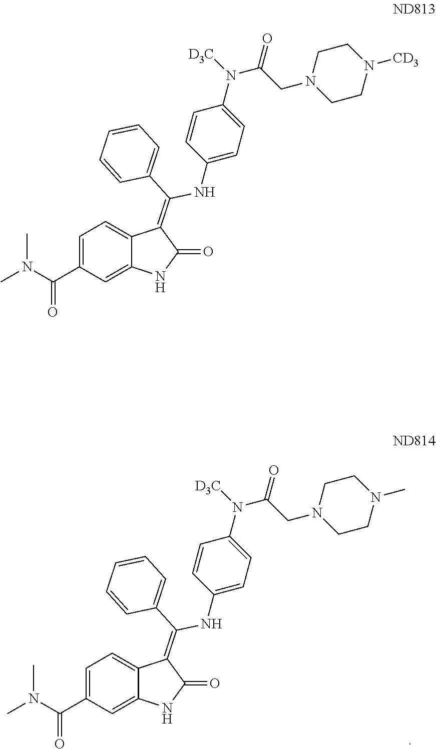

3. The compound or pharmaceutically acceptable salt thereof according to claim 1, wherein the pharmaceutically acceptable salt is a methanesulfonate, ethanesulfonate, benzenesulfonate, benzenesulfonate, phosphate, dextrorotatory camphorsulfonate, hydrochloride, hydrobromide, hydrofluoride, sulfate, nitrate, formate, acetate, propionate, oxalate, malonate, succinate, fumarate, maleate, lactate, malate, tartrate, citrate, picrate, aspartate or glutamate of the compound.

4. The compound or pharmaceutically acceptable salt thereof according to claim 3, wherein the pharmaceutically acceptable salt is an ethanesulfonate salt of the compound.

5. A method of treating or preventing a fibrosis-related disease in a subject in need thereof, comprising administering to the subject a therapeutically effective amount of the compound according to claim 1 or a pharmaceutically acceptable salt thereof.

6. The method according to claim 5, wherein the fibrosis related disease is selected from idiopathic pulmonary fibrosis, giant cell interstitial pneumonia, sarcoidosis, cystic fibrosis, respiratory distress syndrome, drug-induced pulmonary fibrosis, granuloma, scleroderma, interstitial lung disease, pneumoconiosis, silicosis, asbestosis, acute lung injury, cardiac fibrosis, liver cirrhosis, chronic kidney disease, myocardial infarction, heart failure, non-alcoholic fatty liver disease (NASH), covid-19, and lupus erythematosus.

7. A method of treating or preventing diseases related to excessive or abnormal cell proliferation in a subject in need thereof, comprising administering to the subject a therapeutically effective amount of the compound according to claim 1 or a pharmaceutically acceptable salt thereof.

8. The method according to claim 7, wherein diseases related to excessive or abnormal cell proliferation are selected from acute myeloid leukemia, gastric tumors, neuroendocrine tumors, thyroid tumors, melanoma, squamous cell carcinoma, metastatic non-small cell lung cancer, soft tissue sarcoma, pterygium or neovascular eye disease.

9. Avascular endothelial growth factor receptor (VEGFR) inhibitor, comprising the compound according to claim 1 or a pharmaceutically acceptable salt thereof.

10. A fibroblast growth factor receptor (FGFR) inhibitor, comprising the compound according to claim 1 or a pharmaceutically acceptable salt thereof.

11. A platelet-derived growth factor receptor (PDGFR) inhibitor, comprising the compound according to claim 1 or a pharmaceutically acceptable salt thereof.

12. A pharmaceutical composition comprising the compound of claim 1 or a pharmaceutically acceptable salt thereof as an active ingredient and a pharmaceutically acceptable excipient.
